(19) United States
(12) Patent Application Publication
(10) Pub. No.: US 2026/0021306 A1
(43) Pub. Date: Jan. 22, 2026

(54) Using Stimulation Circuitry to Provide DC Offset Compensation in a Stimulator Device Having Tissue Signal Sensing Capability

(71) Applicant: Boston Scientific Neuromodulation Corporation, Valencia, CA (US)

(72) Inventors: Nathan Maas, Simi Valley, CA (US); Joseph M. Bocek, Seattle, WA (US)

(21) Appl. No.: 19/262,488

(22) Filed: Jul. 8, 2025

Related U.S. Application Data
(60) Provisional application No. 63/673,561, filed on Jul. 19, 2024.

(51) Int. Cl. A61N 1/36

(52) U.S. Cl. CPC A61N 1/36139; A61N 1/36062

(57) ABSTRACT

DC offset compensation is provided to equate the DC values of the inputs to sense amp circuitry used to sense tissue signals in a stimulator device. When a DC offset is present at the inputs to the sense amp circuitry, the stimulation circuitry is controlled to remove this DC offset, which can occur in different ways. In one example, charge imbalanced pulses are provided to the inputs of the sense amp circuitry, the stimulation electrodes, or to other electrodes. Control of the stimulation circuitry can occur using a DC offset compensation algorithm programmed into control circuitry of the stimulator device. Using the algorithm, measurements indicative of the DC offset are used to determine charge imbalanced pulses, with the measurements being made by shorting the inputs together, and then releasing the input to reestablish the DC offset.

### CROSS REFERENCE TO RELATED APPLICATIONS

This is a non-provisional filing of U.S. Provisional Patent Application Ser. No. 63/673,561, filed Jul. 19, 2024, which is incorporated herein by reference in its entirety and to which priority is claimed.

### FIELD OF THE INVENTION

This application relates to Implantable Medical Devices (IMDs), and more specifically to circuitry and algorithms to assist with sensing tissue signals such as neural responses in an implantable stimulator device.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) or Deep Brain Stimulation (DBS) system. However, the present invention may find applicability with any stimulator device system.

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12, or some conductive portion of the case, can also comprise an electrode (Ec). In an SCS application, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. In a DBS application, the electrode leads are implanted in the brain through holes in the skull, and lead extension are used to connect the leads to the IPG which is typically implanted under the clavicle (collarbone). In other IPG examples designed for implantation directly at a site requiring stimulation, the IPG can be lead-less, having electrodes 16 instead appearing on the body of the IPG 10 for contacting the patient's tissue. The IPG lead(s) can be integrated with and permanently connected to the IPG 10 in other solutions. SCS therapy can relieve symptoms such as chronic back pain, while DBS therapy can alleviate Parkinsonian symptoms such as tremor and rigidity. IPGs as described should be understood as including External Trial Stimulators (ETSs), which mimic operation of the IPG during trials periods when leads have been implanted in the patient but the IPG has not. See, e.g., U.S. Pat. No. 9,259,574 (disclosing an ETS).

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30i), as shown in the example of FIG. 2A. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as an anode (during its first phase 30a), and thus provides pulses which source a positive current of amplitude +I to the tissue. Electrode E2 has been selected as a cathode (again during first phase 30a), and thus provides pulses which sink a corresponding negative current of amplitude −I from the tissue. This is an example of bipolar stimulation, in which the lead includes one anode pole and one cathode pole. Note that more than one electrode on the lead may be selected to act as an anode electrode to form an anode pole at a given time, and more than one electrode may be selected to act as a cathode to form a cathode pole at a given time, as explained further in U.S. Pat. No. 10,881,859. Stimulation provided by the IPG 10 can also be monopolar. In monopolar stimulation, the lead is programmed with a single pole of a given polarity (e.g., a cathode pole), with the conductive case electrode Ec acting as a return (e.g., an anode pole). Again, more than one electrode on the lead may be active to form the pole during monopolar stimulation.

IPG 10 as mentioned includes stimulation circuitry 28 to form prescribed stimulation at a patient's tissue. FIG. 3 shows an example of stimulation circuitry 28, which includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2A, FIG. 3 shows operation during the first phase 30a in which electrode E1 has been selected as an anode electrode to source current I to the tissue R and E2 has been selected as a cathode electrode to sink current from the tissue. Thus PDAC1 and NDAC2 are digitally programmed to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16. Other stimulation circuitries 28 can also be used in the IPG 10, including ones that includes switching matrices between the electrode nodes ei 39 and the N/PDACs. Sec, e.g., 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519, which are incorporated herein by reference. As explained in these references, ASIC(s) may also contain other circuitry useful in the IPG 10, such as IPG master control circuitry 102 (see FIG. 5), telemetry circuitry (for interfacing off chip with telemetry antennas 27a and/or 27b), circuitry for generating the compliance voltage VH (as explained next), various measurement circuits, etc.

Power for the stimulation circuitry 28 is provided by a compliance voltage VH, as described in further detail in U.S. Patent Application Publications 2013/0289665 and 2018/0071520. The compliance voltage VH may be coupled to the source circuitry (e.g., the PDAC(s)), while ground may be coupled to the sink circuitry (e.g., the NDAC(s)), such that the stimulation circuitry 28 is powered by VH and ground. Other power supply voltages may be used with the PDACs and NDACs, and explained in U.S. Patent Application Publication 2018/0071520, but these aren't shown in FIG. 3 for simplicity.

Preferably, and as described in U.S. Pat. No. 11,040,202, the compliance voltage VH can be produced by a VH regulator 49. VH regulator 49 receives the voltage of the battery 14 (Vbat) and boosts this voltage to a higher value required for the compliance voltage VH. VH regulator 49 can comprise an inductor-based boost converter or a capacitor-based charge pump for example. The regulator 49 can vary the value of VH based on measurements taken from the stimulation circuitry 28 as explained in detail in the '202 patent. Using such measurements allows VH to be established at an energy-efficient level: high enough to form the prescribed current without loading (i.e., without producing less current that prescribed), yet low enough to not needlessly waste power in the stimulation circuitry 28 when forming the prescribed current. In this respect, VH can be variable, and typically ranges from about 5 to 15 Volts.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. While useful, DC-blocking capacitors 38 are not strictly required in all IPG designs and applications.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will (primarily) build up across the DC-blockings capacitors C1 and C2 associated with the electrodes E1 and E2 used to produce the current, giving rise to voltages Vc1 and Vc2 (I=C*dV/dt). During the second pulse phase 30b, when the polarity of the current I is reversed at the selected electrodes E1 and E2, the stored charge on capacitors C1 and C2 is recovered, and thus voltages Vc1 and Vc2 are intended to return to 0V at the end the second pulse phase 30b. Typically, the biphasic pulses used are programmed as charge balanced because the charge of each of the pulse's phases (+Q and −Q) are equal, and thus cancel to zero.

Charge recovery using phases 30a and 30b is said to be “active” because the P/NDACs in stimulation circuitry 28 actively drive a current, in particular during the last phase 30b to recover charge stored after the first phase 30a. However, because the current sources may not be perfectly balanced or other factors, such active charge recovery may not be perfect, and some residual charge may be present in capacitive structures even after phase 30b is completed. Accordingly, the stimulation circuitry 28 can also provide for passive charge recovery. Passive charge recovery is implemented using passive charge recovery switches PRi 41 as shown in FIG. 3. These switches 41 when selected via assertion of control signals <Xi> couple each desired electrode node ei to a passive recovery voltage Vpr established on bus 43. As explained in U.S. Pat. Nos. 10,716,937 and 10,792,491, this allows any stored charge to be recovered through the patient's tissue, R, passive and without the P/NDACs in stimulation circuitry 28 actively driving a current. Control signals <Xi> are usually asserted to cause passive charge recovery after each pulse (e.g., after each last phase 30b) during periods 30c shown in FIG. 2A. Because passive charge recovery involves capacitive discharge through the resistance R of the patient's tissue, such discharge manifests as an exponential decay in current, as shown in FIG. 2A. As also discussed in the '937 patent, each of the passive charge recovery switches 41 can be associated with a variable resistance, and as such each switch 41 can be controlled by a bus of signals <Xi> to control the resistance at which passive charge recovery occurs—i.e., the on resistance of the switches 41 when they are closed.

Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 27a in the IPG 10. The external controller 60 can also have a far-field RF antenna 64b capable of wirelessly communicating with the RF antenna 27b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 27a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 27b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both of the server 86 or the terminal 87.

### SUMMARY

A stimulator device is disclosed, which may comprise: a plurality of electrode nodes, wherein each of the electrode nodes is associated with a different electrode configured to contact a patient's tissue; stimulation circuitry configurable to provide stimulation to one or more first of the plurality of electrode nodes to provide stimulation to the patient's tissue; sense amplifier circuitry comprising a first input and a second input, wherein the sense amplifier circuitry is configurable to receive second of the plurality of electrode nodes at the first and second inputs, wherein the sense amplifier circuitry is configured to sense a tissue signal; and control circuitry configured to short and then release the first and second inputs, and to receive at least one measurement from the sense amp circuitry taken after the release to produce data indicative of a DC offset voltage between the first input and the second input; wherein the control circuitry is further configured to use the data to control the stimulation circuitry to reduce or eliminate the DC offset voltage.

In one example, the stimulator device further comprises a DC-blocking capacitor between each of the electrode nodes and its associated electrode. In one example, the control circuitry is configured to use the data to control the stimulation circuitry to reduce or eliminate the DC offset voltage by issuing an offset removal current at one or more of the electrode nodes. In one example, the offset removal current comprises one or more charge imbalanced pulses. In one example, the offset removal current is issued at one or more of the one or more first electrode nodes. In one example, the offset removal current is issued by the control circuitry by adjusting a calibration of the stimulation circuitry. In one example, the offset removal current is issued at one or more of the one or more second electrode nodes. In one example, the offset removal current is issued at one or more of the plurality of electrode nodes other than the first or second electrode nodes. In one example, at least one of the one or more first electrode nodes is the same as at least one of the one or more second electrode nodes. In one example, all of the one or more first electrode nodes are different from the one or more second electrode nodes. In one example, the sense amplifier circuitry is configured to sense a neural response to the stimulation as the tissue signal. In one example, the control circuitry comprises an algorithm to receive the at least one measurement and to control the stimulation circuitry to reduce or eliminate the DC offset voltage. In one example, the algorithm is configured to iterate by periodically producing the data indicative of the DC offset voltage, and periodically using the data to control the stimulation circuitry to reduce or eliminate the DC offset voltage. In one example, the stimulation is provided as biphasic pulses, and wherein the control circuitry is configured to use the data to control the stimulation circuitry to reduce or eliminate the DC offset voltage by adjusting an interphase interval between the phases of the biphasic pulses. In one example, the control circuitry is configured to use the data to control the stimulation circuitry to reduce or eliminate the DC offset voltage by adjusting an anodic or cathodic current of the stimulation at the one or more first electrode nodes. In one example, the adjustment of the anodic or cathodic current comprises steering at least some of anodic or cathodic current between the one or more first electrode nodes. In one example, the adjustment of the current comprises steering at least some of anodic or cathodic current to a new one or more first electrode nodes. In one example, the control circuitry is configured to short the first and second inputs to a reference voltage. In one example, the control circuitry is configured to receive a first measurement from the sense amp circuitry taken while the first and second inputs are shorted, and a second measurement taken after the release, wherein the data indicative of the DC offset voltage comprises a difference between the first and second measurements. In one example, the control circuitry is configured to receive a plurality of measurements over time from the sense amp circuitry taken after the release, wherein the data indicative of the DC offset voltage varies over time.

A method is disclosed for operating a stimulator device comprising a plurality of electrode nodes, wherein each of the electrode nodes is associated with a different electrode configured to contact a patient's tissue. The method may comprise: providing from stimulation circuitry stimulation to one or more first of the plurality of electrode nodes to provide stimulation to the patient's tissue; using sense amplifier circuitry to sense a tissue signal, the sense amplifier circuitry comprising a first input and a second input, wherein the sense amplifier circuitry receives second of the plurality of electrode nodes at the first and second inputs; shorting and then releasing the first and second inputs, and receiving at least one measurement from the sense amp circuitry taken after the release to produce data indicative of a DC offset voltage between the first input and the second input; and using the data to control the stimulation circuitry to reduce or eliminate the DC offset voltage.

In one example, a DC-blocking capacitor is between each of the electrode nodes and its associated electrode. In one example, the data is used to control the stimulation circuitry to reduce or eliminate the DC offset voltage by issuing an offset removal current at one or more of the electrode nodes. In one example, the offset removal current comprises one or more charge imbalanced pulses. In one example, the offset removal current is issued at one or more of the one or more first electrode nodes. In one example, the data is used to control the stimulation circuitry to reduce or eliminate the DC offset voltage by adjusting a calibration of the stimulation circuitry. In one example, the offset removal current is issued at one or more of the one or more second electrode nodes. In one example, the offset removal current is issued at one or more of the plurality of electrode nodes other than the first or second electrode nodes. In one example, at least one of the one or more first electrode nodes is the same as at least one of the one or more second electrode nodes. In one example, all of the one or more first electrode nodes are different from the one or more second electrode nodes. In one example, the tissue signal comprises a neural response to the stimulation. In one example, at least a portion of the method is controlled by an algorithm programmed into control circuitry of the stimulator device. In one example, the algorithm iterates by periodically producing the data indicative of the DC offset voltage, and periodically uses the data to control the stimulation circuitry to reduce or eliminate the DC offset voltage. In one example, the stimulation is provided as biphasic pulses, and wherein the data is used to control the stimulation circuitry to reduce or eliminate the DC offset voltage by adjusting an interphase interval between the phases of the biphasic pulses. In one example, the data is used to control the stimulation circuitry to reduce or eliminate the DC offset voltage by adjusting an anodic or cathodic current of the stimulation at the one or more first electrode nodes. In one example, the adjustment of the anodic or cathodic current comprises steering at least some of anodic or cathodic current between the one or more first electrode nodes. In one example, the adjustment of the current comprises steering at least some of anodic or cathodic current to a new one or more first electrode nodes. In one example, the first and second inputs are shorted to a reference voltage. In one example, the method further comprises receiving a first measurement from the sense amp circuitry taken while the first and second inputs are shorted, and a second measurement taken after the release, wherein the data indicative of the DC offset voltage comprises a difference between the first and second measurements. In one example, the method receives a plurality of measurements over time from the sense amp circuitry taken after the release, wherein the data indicative of the DC offset voltage varies over time.

A non-transitory computer readable medium is disclosed comprising instructions executable in a stimulator device comprising a plurality of electrode nodes, wherein each of the electrode nodes is associated with a different electrode configured to contact a patient's tissue, wherein the instructions when executed are configured to: provide from stimulation circuitry stimulation to one or more first of the plurality of electrode nodes to provide stimulation to the patient's tissue; use sense amplifier circuitry to sense a tissue signal, the sense amplifier circuitry comprising a first input and a second input, wherein the sense amplifier circuitry is configurable to receive second of the plurality of electrode nodes at the first and second inputs; short and then release the first and second inputs, and receive at least one measurement from the sense amp circuitry taken after the release to produce data indicative of a DC offset voltage between the first input and the second input; and use the data to control the stimulation circuitry to reduce or eliminate the DC offset voltage.

A stimulator device is disclosed, which may comprise: a plurality of electrode nodes, wherein each of the electrode nodes is associated with a different electrode configured to contact a patient's tissue; stimulation circuitry configurable to provide stimulation to one or more first of the plurality of electrode nodes to provide stimulation to the patient's tissue; sense amplifier circuitry comprising a first input and a second input, wherein the sense amplifier circuitry is configurable to receive second of the plurality of electrode nodes at the first and second inputs, wherein the sense amplifier circuitry is configured to sense a tissue signal; wherein the sense amplifier circuitry is further configured to produce data indicative of a DC offset voltage between the first input and the second input; and control circuitry configured to use the data to control the stimulation circuitry to issue compensation at one or more of the first plurality of electrode nodes to reduce or eliminate the DC offset voltage.

In one example, the stimulator device further comprises a DC-blocking capacitor between each of the electrode nodes and its associated electrode. In one example, the compensation comprises an offset removal current issued at the one or more first electrode nodes. In one example, the offset removal current comprises one or more charge imbalanced pulses. In one example, the offset removal current is issued by the control circuitry by adjusting a calibration of the stimulation circuitry. In one example, at least one of the one or more first electrode nodes is the same as at least one of the one or more second electrode nodes. In one example, all of the one or more first electrode nodes are different from the one or more second electrode nodes. In one example, the sense amplifier circuitry is configured to sense a neural response to the stimulation as the tissue signal. In one example, the control circuitry comprises an algorithm to receive the data indicative of the DC offset voltage and to control the stimulation circuitry to reduce or eliminate the DC offset voltage. In one example, the algorithm is configured to iterate by periodically producing the data indicative of the DC offset voltage, and periodically using the data to control the stimulation circuitry to reduce or eliminate the DC offset voltage. In one example, the stimulation is provided as biphasic pulses, and wherein the compensation comprises adjusting an interphase interval between the phases of the biphasic pulses. In one example, the compensation comprises adjusting an anodic or cathodic current of the stimulation at the one or more first electrode nodes. In one example, the adjustment of the anodic or cathodic current comprises steering at least some of anodic or cathodic current between the one or more first electrode nodes. In one example, the adjustment of the current comprises steering at least some of anodic or cathodic current to a new one or more first electrode nodes. In one example, the control circuitry is further configured to short and then release the first and second inputs, and to receive at least one measurement from the sense amp circuitry taken after the release to produce the data indicative of the DC offset voltage between the first input and the second input. In one example, the control circuitry is configured to short the first and second inputs to a reference voltage. In one example, the control circuitry is configured to receive a first measurement from the sense amp circuitry taken while the first and second inputs are shorted, and a second measurement taken after the release, wherein the data indicative of the DC offset voltage comprises a difference between the first and second measurements. In one example, the control circuitry is configured to receive a plurality of measurements over time from the sense amp circuitry taken after the release, wherein the data indicative of the DC offset voltage varies over time.

A method is disclosed for operating a stimulator device comprising a plurality of electrode nodes, wherein each of the electrode nodes is associated with a different electrode configured to contact a patient's tissue. The method may comprise: providing from stimulation circuitry stimulation to one or more first of the plurality of electrode nodes to provide stimulation to the patient's tissue; using sense amplifier circuitry to sense a tissue signal, the sense amplifier circuitry comprising a first input and a second input, wherein the sense amplifier circuitry receives second of the plurality of electrode nodes at the first and second inputs; producing data indicative of a DC offset voltage between the first input and the second input; and using the data to control the stimulation circuitry to issue compensation at one or more of the first plurality of electrode nodes to reduce or eliminate the DC offset voltage.

In one example, a DC-blocking capacitor is between each of the electrode nodes and its associated electrode. In one example, the compensation comprises issuing an offset removal current at the one or more first electrode nodes. In one example, the offset removal current comprises one or more charge imbalanced pulses. In one example, the data is used to control the stimulation circuitry to reduce or eliminate the DC offset voltage by adjusting a calibration of the stimulation circuitry. In one example, at least one of the one or more first electrode nodes is the same as at least one of the one or more second electrode nodes. In one example, all of the one or more first electrode nodes are different from the one or more second electrode nodes. In one example, the tissue signal comprises a neural response to the stimulation. In one example, at least a portion of the method is controlled by an algorithm programmed into control circuitry of the stimulator device. In one example, the algorithm iterates by periodically producing the data indicative of the DC offset voltage, and periodically uses the data to control the stimulation circuitry to reduce or eliminate the DC offset voltage. In one example, the stimulation is provided as biphasic pulses, and wherein the compensation comprises adjusting an interphase interval between the phases of the biphasic pulses. In one example, the compensation comprises adjusting an anodic or cathodic current of the stimulation at the one or more first electrode nodes. In one example, the adjustment of the anodic or cathodic current comprises steering at least some of anodic or cathodic current between the one or more first electrode nodes. In one example, the adjustment of the current comprises steering at least some of anodic or cathodic current to a new one or more first electrode nodes. In one example, the method further comprises shorting and then releasing the first and second inputs, and receiving at least one measurement from the sense amp circuitry taken after the release to produce the data indicative of the DC offset voltage between the first input and the second input. In one example, the first and second inputs are shorted to a reference voltage. In one example, the method further comprises receiving a first measurement from the sense amp circuitry taken while the first and second inputs are shorted, and receiving a second measurement taken after the release, wherein the data indicative of the DC offset voltage comprises a difference between the first and second measurements. In one example, the method receives a plurality of the measurements over time from the sense amp circuitry taken after the release, wherein the data indicative of the DC offset voltage varies over time.

A non-transitory computer readable medium is disclosed comprising instructions executable in a stimulator device comprising a plurality of electrode nodes, wherein each of the electrode nodes is associated with a different electrode configured to contact a patient's tissue, wherein the instructions when executed are configured to: provide from stimulation circuitry stimulation to one or more first of the plurality of electrode nodes to provide stimulation to the patient's tissue; use sense amplifier circuitry to sense a tissue signal, the sense amplifier circuitry comprising a first input and a second input, wherein the sense amplifier circuitry receives second of the plurality of electrode nodes at the first and second inputs; produce data indicative of a DC offset voltage between the first input and the second input; and use the data to control the stimulation circuitry to issue compensation at one or more of the first plurality of electrode nodes to reduce or eliminate the DC offset voltage.

A stimulator device is disclosed, which may comprise: a plurality of electrode nodes, wherein each of the electrode nodes is associated with a different electrode configured to contact a patient's tissue; stimulation circuitry configurable to provide stimulation to one or more first of the plurality of electrode nodes to provide stimulation to the patient's tissue; sense amplifier circuitry comprising a first input and a second input, wherein the sense amplifier circuitry is configurable to receive second of the plurality of electrode nodes at the first and second inputs, wherein the sense amplifier circuitry is configured to sense a tissue signal; wherein the sense amplifier circuitry is further configured to produce data indicative of a DC offset voltage between the first input and the second input; and control circuitry configured to use the data to control the stimulation circuitry to issue compensation at one or more of the plurality of electrode nodes other than the first or second electrode nodes to reduce or eliminate the DC offset voltage.

In one example, the stimulator device further comprises a DC-blocking capacitor between each of the electrode nodes and its associated electrode. In one example, the compensation comprises an offset removal current issued at the one or more other electrode nodes. In one example, the offset removal current comprises one or more charge imbalanced pulses. In one example, at least one of the one or more first electrode nodes is the same as at least one of the one or more second electrode nodes. In one example, all of the one or more first electrode nodes are different from the one or more second electrode nodes. In one example, the sense amplifier circuitry is configured to sense a neural response to the stimulation as the tissue signal. In one example, the control circuitry comprises an algorithm to receive the data indicative of the DC offset voltage and to control the stimulation circuitry to reduce or eliminate the DC offset voltage. In one example, the algorithm is configured to iterate by periodically producing the data indicative of the DC offset voltage, and periodically using the data to control the stimulation circuitry to reduce or eliminate the DC offset voltage. In one example, the compensation comprises steering an anodic or cathodic current of the stimulation to the one or more other electrode nodes. In one example, the control circuitry is further configured to short and then release the first and second inputs, and to receive at least one measurement from the sense amp circuitry taken after the release to produce the data indicative of the DC offset voltage between the first input and the second input. In one example, the control circuitry is configured to short the first and second inputs to a reference voltage. In one example, the control circuitry is configured to receive a first measurement from the sense amp circuitry taken while the first and second inputs are shorted, and a second measurement taken after the release, wherein the data indicative of the DC offset voltage comprises a difference between the first and second measurements. In one example, the control circuitry is configured to receive a plurality of measurements over time from the sense amp circuitry taken after the release, wherein the data indicative of the DC offset voltage varies over time.

A method is disclosed for operating a stimulator device comprising a plurality of electrode nodes, wherein each of the electrode nodes is associated with a different electrode configured to contact a patient's tissue. The method may comprise: providing from stimulation circuitry stimulation to one or more first of the plurality of electrode nodes to provide stimulation to the patient's tissue; using sense amplifier circuitry to sense a tissue signal, the sense amplifier circuitry comprising a first input and a second input, wherein the sense amplifier circuitry receives second of the plurality of electrode nodes at the first and second inputs; producing data indicative of a DC offset voltage between the first input and the second input; and using the data to control the stimulation circuitry to issue compensation one or more of the plurality of electrode nodes other than the first or second electrode nodes to reduce or eliminate the DC offset voltage.

In one example, a DC-blocking capacitor is between each of the electrode nodes and its associated electrode. In one example, the compensation comprises issuing an offset removal current at the one or more other electrode nodes. In one example, the offset removal current comprises one or more charge imbalanced pulses. In one example, at least one of the one or more first electrode nodes is the same as at least one of the one or more second electrode nodes. In one example, all of the one or more first electrode nodes are different from the one or more second electrode nodes. In one example, the tissue signal comprises a neural response to the stimulation. In one example, at least a portion of the method is controlled by an algorithm programmed into control circuitry of the stimulator device. In one example, the algorithm iterates by periodically producing the data indicative of the DC offset voltage, and periodically uses the data to control the stimulation circuitry to reduce or eliminate the DC offset voltage. In one example, the compensation comprises steering an anodic or cathodic current of the stimulation to the one or more other electrode nodes. In one example, the method further comprises shorting and then releasing the first and second inputs, and receiving at least one measurement from the sense amp circuitry taken after the release to produce the data indicative of the DC offset voltage between the first input and the second input. In one example, the first and second inputs are shorted to a reference voltage. In one example, the method further comprises receiving a first measurement from the sense amp circuitry taken while the first and second inputs are shorted, and receiving a second measurement taken after the release, wherein the data indicative of the DC offset voltage comprises a difference between the first and second measurements. In one example, the method comprises receiving a plurality of the measurements over time from the sense amp circuitry taken after the release, wherein the data indicative of the DC offset voltage varies over time.

A non-transitory computer readable medium is disclosed comprising instructions executable in a stimulator device comprising a plurality of electrode nodes, wherein each of the electrode nodes is associated with a different electrode configured to contact a patient's tissue, wherein the instructions when executed are configured to: provide from stimulation circuitry stimulation to one or more first of the plurality of electrode nodes to provide stimulation to the patient's tissue; use sense amplifier circuitry to sense a tissue signal, the sense amplifier circuitry comprising a first input and a second input, wherein the sense amplifier circuitry receives second of the plurality of electrode nodes at the first and second inputs; produce data indicative of a DC offset voltage between the first input and the second input; and use the data to control the stimulation circuitry to issue compensation one or more of the plurality of electrode nodes other than the first or second electrode nodes to reduce or eliminate the DC offset voltage.

### BRIEF DESCRIPTION OF THE DRAWINGS

FIG. 1 shows an Implantable Pulse Generator (IPG), in accordance with the prior art.

FIGS. 2A and 2B show an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5 shows an IPG having tissue signal sensing capability.

FIG. 6 shows sense amp circuitry useable in an IPG having tissue signal sensing capability.

FIG. 7 shows stimulation producing a tissue signal, and the sensing of that tissue signal at at least one electrode of the IPG.

FIG. 8 shows how a DC offset can cause saturation or clipping of digitized waveforms of a tissue signal, and how DC offset compensation mitigates this.

FIGS. 9A-9C show different approaches to DC offset compensation in which the stimulation circuitry is controlled by a DC offset compensation algorithm to provide compensation at the sensing electrodes (FIG. 9A), at the stimulation electrodes (FIG. 9B), or to any other electrode (FIG. 9C). FIG. 9D shows that these different types of electrodes may not be discrete from each other and may overlap.

FIGS. 10A and 10B show different manners by which DC offset compensation can be affected.

FIGS. 11A and 11B show a first example of the DC offset compensation algorithm in which compensation is provided by providing an offset removal current to the inputs of the sense amp at the sensing electrodes.

FIG. 12 shows a second example of the DC offset compensation algorithm in which compensation is provided by providing an offset removal current to the inputs of the sense amp at the sensing electrodes.

FIGS. 13A and 13B show a third example of the DC offset compensation algorithm in which compensation is provided by calibrating the stimulation circuitry at the stimulation electrodes.

FIG. 14 shows a fourth example of the DC offset compensation algorithm in which compensation is provided by adjusting the interpulse interval between phases of the stimulation pulse.

FIG. 15 shows a fifth example of the DC offset compensation algorithm in which compensation is provided by adjusting the fractionalization of the current at the stimulation electrodes.

FIGS. 16A and 16B show a sixth example of the DC offset compensation algorithm in which the charge of the needed offset removal current is computed.

FIGS. 17A and 17B show a seventh example of the DC offset compensation algorithm in which the needed offset removal current is formed as a single shaped pulse.

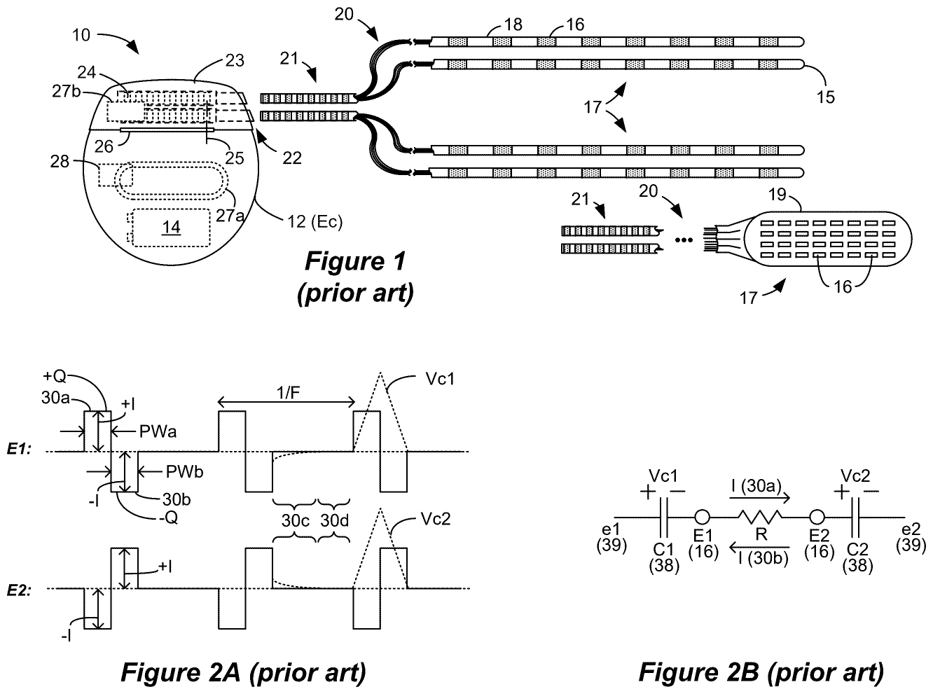

### DETAILED DESCRIPTION

An increasingly interesting development in pulse generator systems is the addition of sensing capability to complement the stimulation that such systems provide. For example, it can be beneficial to sense a neural response produced by neural tissue that has received stimulation from an IPG. U.S. Pat. No. 10,406,368 shows an example where sensing of neural responses is useful in an SCS context, and in particular discusses the sensing of Evoked Compound Action Potentials, or “ECAPs,” in a patient's spinal cord. U.S. Patent Application Publication 2022/0040486 shows an example where sensing of neural responses is useful in a DBS context, and discusses the sensing of Evoked Resonant Neural Activity, or “ERNA,” in a patient's brain. Sensing can also be used to sense local field potentials (LFPs) and other voltages in the tissue, such as stimulation artifacts. See, e.g., U.S. Patent Application Publication 2022/0323764. Collectively, all of these sensed signals comprise tissue signals.

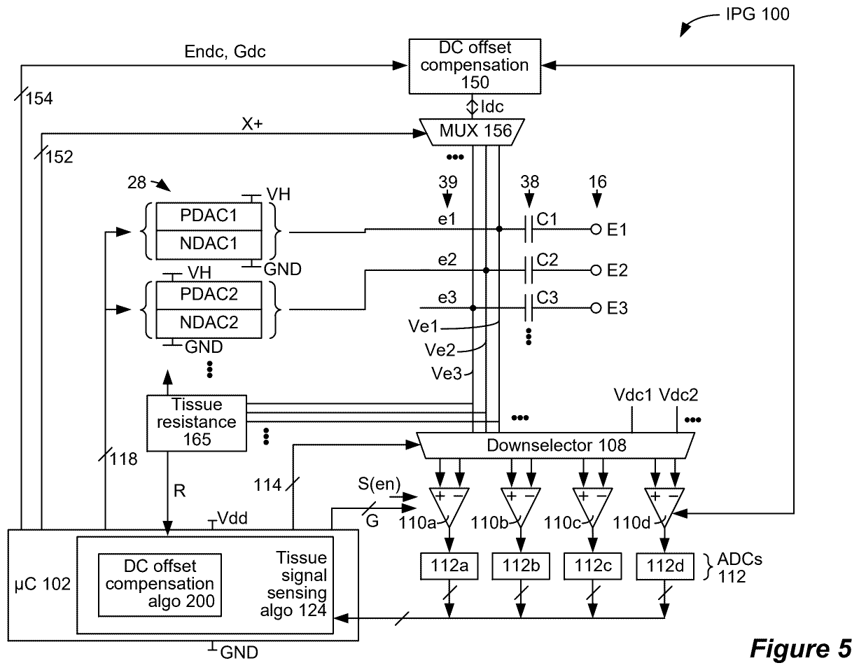

FIG. 5 shows basic circuitry for an IPG 100 having tissue signal sensing capability. The IPG 100 includes control circuitry 102, which may comprise a microcontroller, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs) in the IPG 100 as described earlier, which ASIC(s) may additionally include the other circuitry shown in FIG. 5.

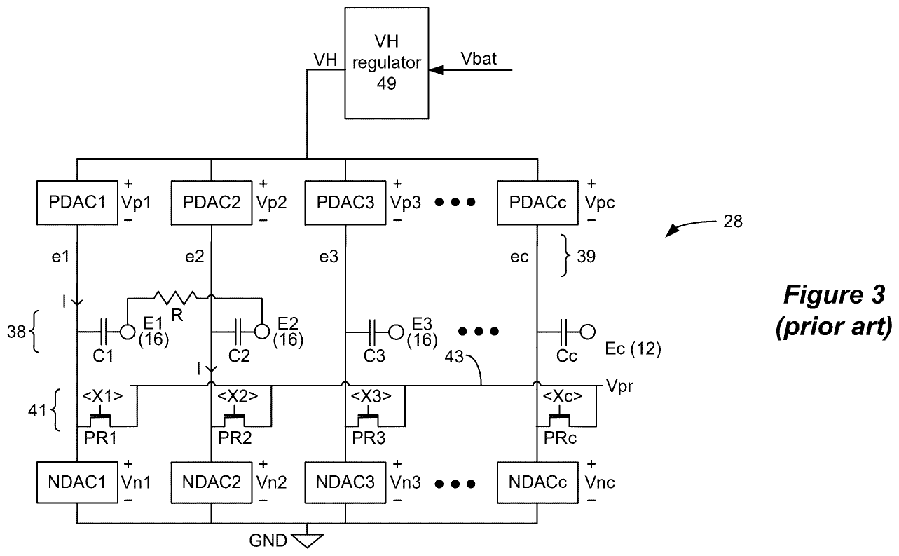

FIG. 5 includes the stimulation circuitry 28 described earlier (FIG. 3), including one or more DACs (PDACs and NDACs). A bus 118 provides digital control signals to the DACs to produce currents or voltages of prescribed amplitudes and with the correct timing at the electrodes selected for stimulation. The current paths to the electrodes include the DC-blocking capacitors 38 described earlier, which separate the electrode nodes 39 from the electrodes 16.

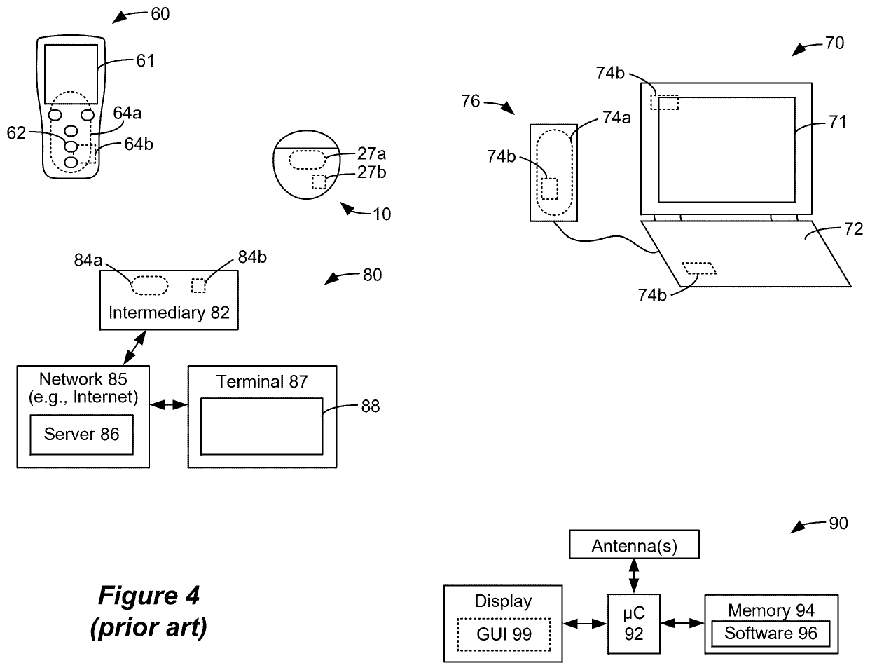

FIG. 5 also shows circuitry used for sensing. As shown, the electrode nodes 39 (Ve1, Ve2, etc.) are input to a downselector 108, which is used to select one or more sensing electrodes. The downselector 108 comprises a multiplexer controlled by a bus 114 issued from the control circuitry 102. The downselector 108 can also operate to select one or more DC voltages (Vdc1, Vdc2, etc.), as is useful in single-ended sensing. The DC voltages can comprise any DC voltage produced within the IPG, such as ground, the voltage of the battery (Vbat), the compliance voltage VH, a power supply voltage (Vdd), or some fraction of these (such as VH/2 or Vdd/2). The sensing electrode(s) and/or DC voltages selected via bus 114 can be determined automatically by control circuitry 102 and/or a tissue signal sensing algorithm 124, as described further below. However, the sensing electrode(s) may also be selected by the user (e.g., a clinician) via an external system 60, 70 or 80 (FIG. 4). Downselector 108 is described further in U.S. patent application Ser. No. 19/055,361, filed Feb. 17, 2025, which is incorporated herein by reference in its entirety.

Electrodes nodes 39 and/or DC voltages selected by the downselector 108 are provided to sense amplifier (amp) circuitry 110 comprising one or more sense amps 110x, each having a positive (+) and negative (−) input. In the example shown, there are four sense amps 110a-110d, which allows sensing to occur on four sensing channels simultaneously. Other implementations may use only a single sense amp. Sensing can occur differentially using two sensing electrodes (two selected electrode nodes 39) that are provided to the positive and negative inputs of a sense amp, or in a single-ended fashion using a single sensing electrode (positive input) and one of the DC voltages (negative input).

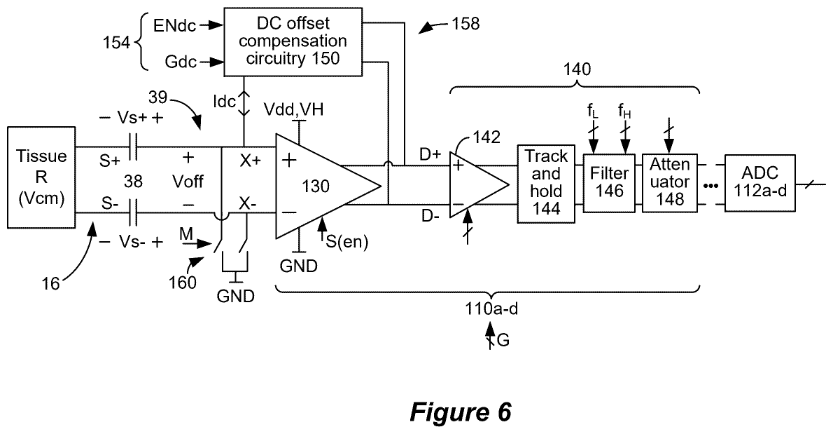

As shown in FIG. 6, the sense amps 110a-d may include a differential amplifier (diff amp) 130 whose gain is programmable, and may also include front-end circuitry at its inputs and/or back-end circuitry 140 at its outputs. Such back-end circuitry 140 can include one or more additional diff amps 142 (allowing for further gain adjustments), track-and-hold circuitry 144, one or more filters 146, one or more attenuators 148, input and output voltage protection (not shown), voltage assessment logic (not shown), and the like. Collectively, the gain of the sense amps 110 (e.g., the diff amp(s) 130 and/or 142) is denoted G, which is programmable by the control circuitry 102 and/or the tissue signal sensing algorithm 124. Further details of circuitry useable for the sense amps 110a-d is disclosed in U.S. Pat. No. 11,633,138 and U.S. Patent Application Publication 2023/0173273, which are incorporated herein by reference in their entireties. As shown, the diff amp 130 preferably has a differential input X+/X− and a differential output D+/D−. As disclosed in the '273 Publication, diff amp 130 can comprise a low-voltage diff amp (powered by Vdd), a high-voltage diff amp (powered by VH), or both, allowing use of either amp to be selected.

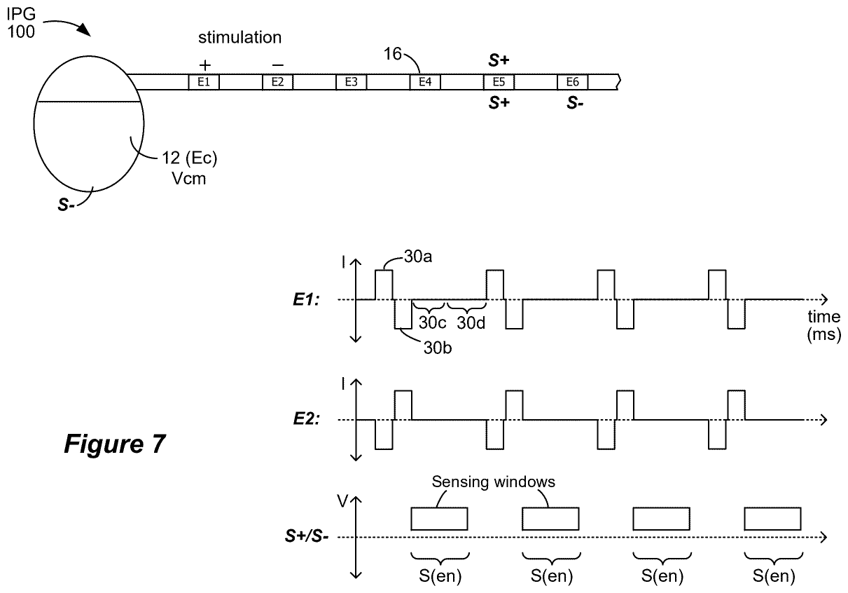

If differential sensing is used, as is more common and as shown in FIG. 7, two electrodes (e.g., E5 and E6) are selected as sensing electrodes (S+ and S−) by the downselector 108, with one electrode (e.g., E5) provided to the positive input X+ of the sense amp circuitry 110, and the other (e.g., E6) provided to the negative input X−. In this example, the sensing electrodes S+ and S− are located on the lead and relatively close to each other, which is particularly useful when sensing ECAPs in a SCS application. The selected sensing electrodes S+ and S− may also be distant from one another. For example, a single lead-based electrode (e.g., E5) may be selected as a sensing electrode (S+), with the more-distant case electrode Ec acting as a sensing reference (S−) as also shown in FIG. 7. Use of the case electrode, or another distant electrode, as a sensing reference is particularly useful when sensing ERNA responses in a DBS application, as discussed further in U.S. Patent Application Publication 2024/0335655. As explained in U.S. Patent Application Publication 2021/0236829, differential sensing can be useful to cancel any common mode DC voltages present in the tissue and reflected at the electrodes, such as voltages created by the stimulation itself.

The selected sensing electrode(s) can be determined in light of the stimulation that is being provided. In this regard, sensing electrodes may be selected near enough to the electrodes providing stimulation (e.g., E1 and E2) to allow for proper tissue signal sensing, but far enough from the stimulation (e.g., E5 and E6) that the stimulation doesn't substantially interfere with tissue signal sensing. See, e.g., U.S. Patent Application Publication 2020/0155019. This is particularly useful when sensing ECAP neural responses during Spinal Cord Stimulation (SCS). However, this is not strictly required. In some instances it can be beneficial to use at least one sensing electrode that is also used to provide stimulation, for example when sensing ERNAs in a DBS context.

As shown in FIG. 7, sensing at the selected sensing electrodes usually occurs after the issuance of the stimulation pulses during sensing windows. The duration of these sensing windows are preferably long enough to allow the sense amp circuitry 110 to sense the tissue signal as a function of time, and suitable durations can depend on the duration of the tissue signals being sensed. Although FIG. 6 shows the issuance of a sensing window after each therapeutic stimulation pulse, this is not strictly required, and therapeutic stimulation pulses can be provided to a patient from time to time without need of sensing. Because sensed tissue signals may be small and difficult to resolve, a number of sensed tissues signals may taken and averaged in the tissue signal sensing algorithm 124 described below. The timing of the sensing windows can be controlled with an enable signal, S(en), which enables sensing at the sense amp circuitry 110. The timing of the sensing windows is programmable, and may be controlled to occur during quiet periods 30d between the pulses. However, sensing of tissue signals such as neural responses can also be achieved during active stimulation (e.g., during phases 30a and/or 30b) or during the provision of passive charge recovery (phase 30c). Sec, e.g., U.S. Pat. No. 11,259,733; U.S. Patent Application Publication 2022/0233866.

Referring again to FIGS. 5 and 6, the analog waveform(s) output by the sense amps 110a-110d are preferably converted to digital signals by one or more Analog-to-Digital converters (ADC) 112a-d, and input to the tissue signal sensing algorithm 124 in the IPG's control circuitry 102. The ADCs 112 can be included within the control circuitry 102's input stage as well, and can comprise part of the sense amp circuitry. The tissue signal sensing algorithm 124 can evaluate the sensed signals, and take appropriate actions as a result. For example, the sensing algorithm 124 may change the stimulation in accordance with a sensed tissue signal, such as an ECAP or ERNA signal, and can issue new control signals via bus 118 to change operation of the stimulation circuitry 28 to affect better treatment for the patient. The sensing algorithm 124 may also cause the selection of new sensing electrode(s), which can be affected by issuing new control signals on bus 114. The tissue signal sensing algorithm 124 can also include a DC offset compensation algorithm 200, which is a focus of this disclosure, and which is discussed in detail further below. Algorithm 200 can also be separate from algorithm 124.

Tissue signals such as neural responses to stimulation are typically small-amplitude AC signals on the order of micro Volts or milliVolts, which can make sensing difficult. For one, the tissue signal being sensed may coincide in time with a stimulation artifact resulting from the electromagnetic field that forms in the tissue as a result of the stimulation, as explained in U.S. Pat. No. 11,633,138. This issue can be mitigated by including tissue biasing circuitry in the IPG 100 that holds the tissue at a common mode voltage, Vcm. This common mode voltage Vcm is preferably established at the conductive case electrode Ec, but can be established at other electrodes as well. See, e.g., U.S. Pat. No. 11,040,202; U.S. Patent Application Publication 2023/0138443.

The presence of a DC offset voltage at the inputs to the relevant sense amp 110 can also complicate sensing. This DC offset is denoted Voff in FIG. 6, and comprises a difference in the DC voltages present at the inputs, which may be positive or negative depending whether the voltage at X+ is higher or lower than the voltage at X−. There are several reasons why a DC offset Voff may be present at the inputs. DC offset Voff may result because the DC-blocking capacitors 38 at the inputs X+/X− are differently charged, as represented by Vs+ and Vs−. Ideally, charge recovery mechanisms discussed earlier-such as the use of biphasic pulses for active charge recovery, and/or the use of passive charge recovery-would mitigate this concern by completely removing charge from these capacitors. However, such charge recovery mechanisms may not operate perfectly and a residual charge (voltage) imbalance may remain on the capacitors. This is especially true given that the capacitance values of the DC-blocking capacitors 38 may not be exactly equal. Even if the DC-blocking capacitors 38 are completely discharged, other mechanisms may cause an inherent DC imbalance at the inputs X+ and X− to the sense amp 110. For example, the diff amp 130 in the sense amp 110 may not be ideal, and may comprise an inherent DC offset at input inputs X+ and X−. Electro-chemical effects at the interface between the electrodes 16 and the tissue can also induce a small DC voltage offset at the inputs to the sense amp 110, particularly if the electrodes 16 input to the sense amp circuitry have different areas or compositions. Still further, a DC offset may form due to the presence of electric fields in the tissue, including those created during the provision of stimulation pulses. Such electric fields may present as different voltages at the sensing electrodes with different magnitudes, and hence charge the capacitors 38 at the sensing electrodes to different degrees, thus causing a DC offset to form.

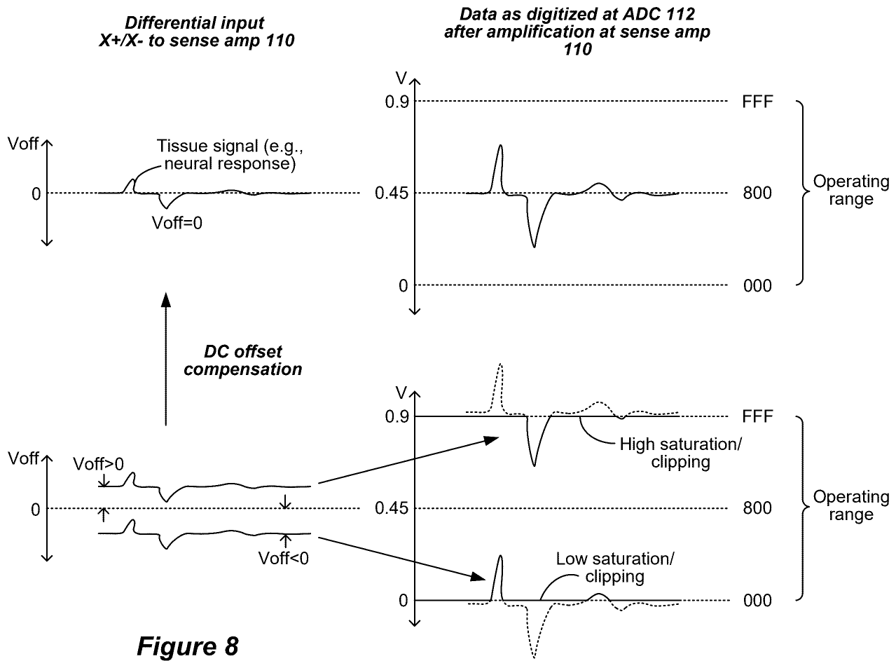

Regardless of the reason that a DC offset Voff might be present, this offset is generally unwanted and undesirable to sense in its own right, because the goal of sensing is typically to sense and amplify only the AC tissue signal. However, any DC offset Voff would also be amplified at the sense amp 110, which is generally undesired for the reasons shown in FIG. 8. The top shows desired sensing of an AC tissue signal when the DC offset Voff is zero. The AC tissue signal is differentially provided to the inputs X+/X− of the sense amp 110 as shown to the left. The result, after amplification (gain G) by the sense amp 110 and digitization at the relevant ADC 112, is shown to the left. In this example, it is assumed that the ADC 112 is a 12-bit ADC that will assign a digital value of 000 to FFF (in hexadecimal, or 000000000000 to 111111111111 in binary) to each of the voltage values in the amplified tissue signal, in accordance with the sampling rate of the ADC. This defines an operating range for the ADC 112, which may be from 0.0 V (000H) to 0.9V (FFFH) in one example. Because Voff is equal to 0 at the top of the FIG. 8, it can be seen that the amplified and digitized tissue signal wholly fits within the operating range, and is roughly centered around the midpoint (0.45V or 800H) of that range. (In this regard, notice Voff=0V can be scaled either by analog or digital circuitry to be at the center of the operating range of the ADC 112). As such, the tissue signal is validly sensed by the ADC 112, and thus the tissue signal sensing algorithm 124 may validly consider this signal.

The bottom of FIG. 8 shows the sensing of a tissue signal when the DC offset Voff is not zero. Notice if Voff is positive that the resulting digitized signal is shifted upwards in the operating range of the ADC 112, because the sense amp 110 has amplified both the DC (Voff) and AC (tissue) signals present at the inputs X+/X−. This may cause the sensed signal, at least in part, to exceed the maximum of the operating range of the ADC 112 (0.9V or FFFH), which clips the sensed signal. Even before the sensed tissue signal is digitized, a positive DC offset Voff may also cause the output (D+/D−, FIG. 6) of the sense amp 110 (its diff amp 130) to saturate, such that its output is pinned to the power supply voltage (VH or Vdd) of the amplifier, which may also result in clipping. In either case, the presence of Voff>0 can hamper resolution of the AC tissue signal, especially if Voff is particularly large (and/or if the gain G is large). The same is true if the DC offset Voff is significantly negative. This would shift the resulting digitized signal downward in the operating range of the ADC 112, possibly to the minimum of the operating range (0.0V or 000H), and again this may occur because the output of the sense amp circuitry 110 is saturated (at ground). In either case, the presence of a DC offset Voff can impact the sensing of the tissue signal.

Because the DC offset Voff is generally not desirable to sense and hampers the sensing of the AC tissue signal, the art has provided techniques to try and ameliorate this offset. First, and as shown in FIGS. 5 and 6, the IPG 100 (its ASIC) may include dedicated DC offset compensation circuitry 150. The details of compensation circuitry 150 are described in detail in the above-incorporated U.S. Patent Application Publication 2023/0173273. Briefly, compensation circuitry 150 operates by measuring (e.g.) the differential output D+/D− of the diff amp 130 in the sense amp circuitry 110, and providing a current Idc to one of the inputs (e.g., X+) to the sense amp circuitry 110. Idc can, depending on the measurement, be positive or negative, and can charge or discharge the DC blocking capacitor 38 (i.e., change Vs+) at X+. Through feedback, this will eventually bring any DC offset Voff towards zero (presumably to a point where Vs+=Vs−), which improves sensing as already explained with reference to FIG. 8. However, Idc may be too small, and hence may take too long to equilibrate Voff towards zero. Further, compensation circuitry 150 and control must be provided for (e.g., on the ASIC), which complicates design.

Recognizing this, the inventors have devised solutions to provide DC offset compensation that do not rely solely on use of a discrete DC offset compensation circuitry such as 150 (although circuitry 150 can also be used in conjunction with the disclosed solutions). Instead, when a DC offset Voff is present at the inputs to the sense amp circuitry 110, the stimulation circuitry 28 is controlled to remove this DC offset in accordance with a DC offset algorithm. In one example, this algorithm provides an offset removal current Ix, which is provided by the stimulation circuitry 28 otherwise normally present and used to provide a stimulation current to selected stimulation electrodes. See also U.S. Patent Application Publication 2024/0055021, disclosing a technique that also uses the stimulation circuitry 28 to remove DC offset compensation, which is incorporated herein by reference in its entirety. This offset removal current Ix may comprise one or more charge imbalanced pulses that are either net cathodic (−Q) or net anodic (+Q). This offset removal current Ix can be provided to one or both of the sensing electrodes, any one or more of the stimulation electrodes, or still other electrodes that are not being used for stimulation or sensing.

DC offset compensation can also be affected in DC compensation algorithm by making other adjustments to the stimulation circuitry 28. For example, an interpulse interval between two phases of a biphasic pulse that provides the stimulation current can be adjusted. In another example, a calibration of stimulation circuitry (the PDAC and NDACs) used to provide the stimulation current can be adjusted. In yet another example, the electrodes selected to provide stimulation can be changed, such as by steering some of the current from selected stimulation electrodes to another new electrode (i.e., by fractionalizing the current).

As discussed further below, measurements indicative of the DC offset are made with the sense amp 110 and ADC 112 and are interpreted by the DC offset compensation algorithm to determine when, and to what extent, DC offset compensation is necessary. These measurements can be taken at discrete (e.g., two) points in time after provision of a stimulation pulse, as explained below, with the algorithm taking any of the actions described above to remediate the offset. Alternatively, the measurements can be taken continuously as a function of time, with the algorithm calculating and providing a compensation pulse with a charge necessary to remove the offset. As discussed further below, these measurements involve shorting the inputs X+ and X−, and then releasing them (letting them float) to understand the magnitude and polarity of Voff.

The DC offset algorithm can be programmed into the IPG's control circuitry 102. The DC offset compensation algorithm can operate as a program within the tissue signal sensing algorithm 124 (FIG. 5), or may comprise a separate program in the control circuitry 102. The DC offset algorithm, like other algorithms operable in the IPG 100, can comprise instructions fixed in a non-transitory computer readable medium, such as a solid-state memory (e.g., control circuitry 102), optical or magnetic disk, and the like. These media may be within the IPG 100, or stored on external systems in manner downloadable to the IPG, such as on various Internet servers and the like, as discussed earlier with reference to FIG. 4.

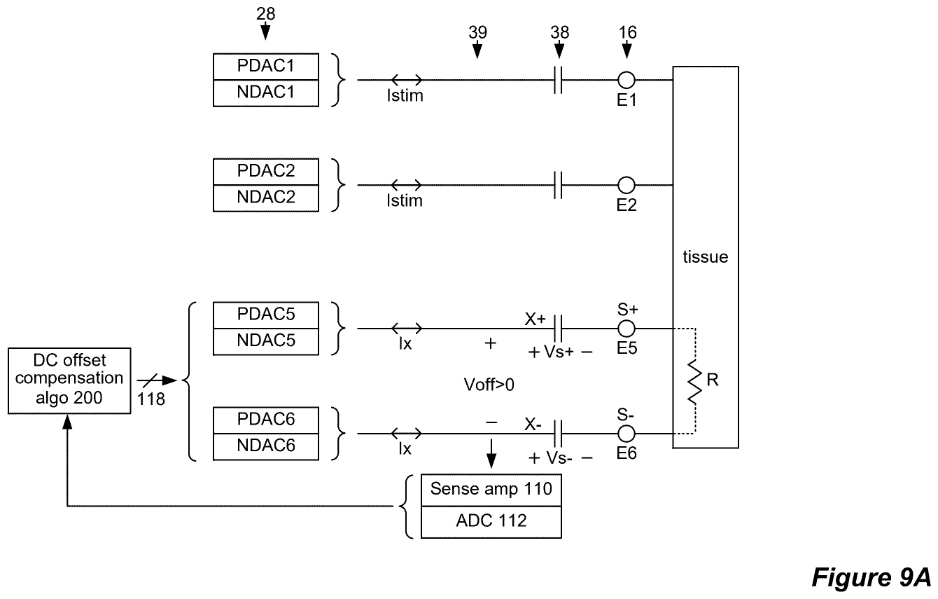

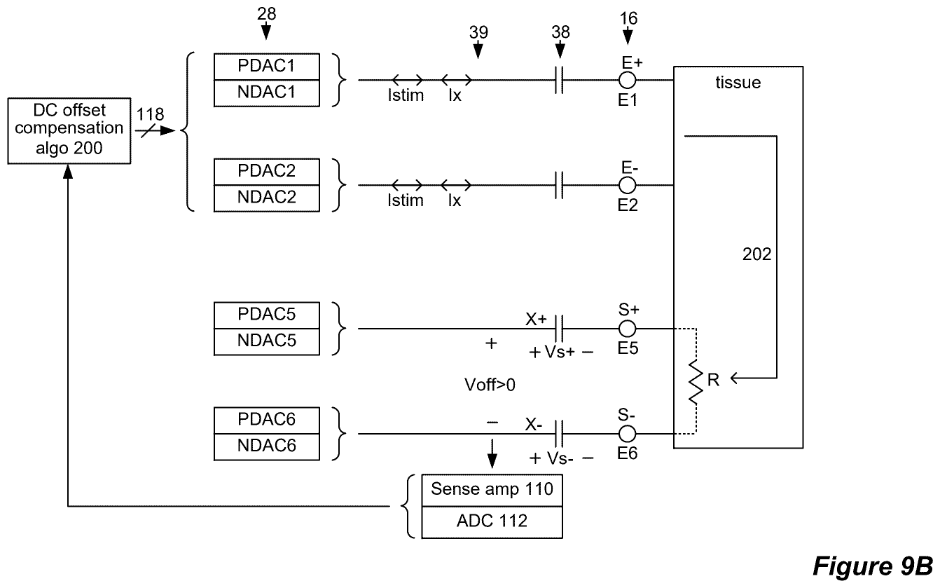

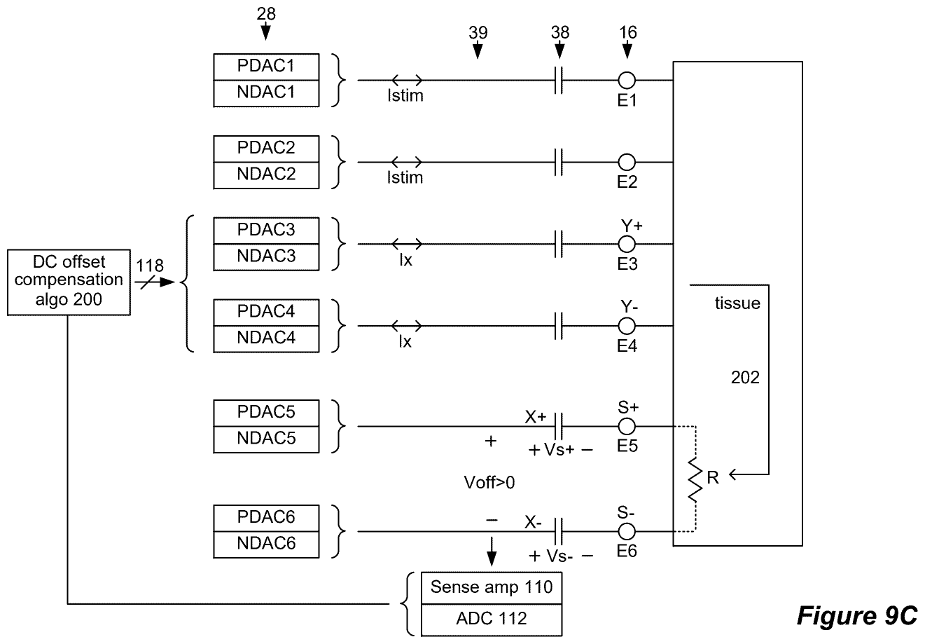

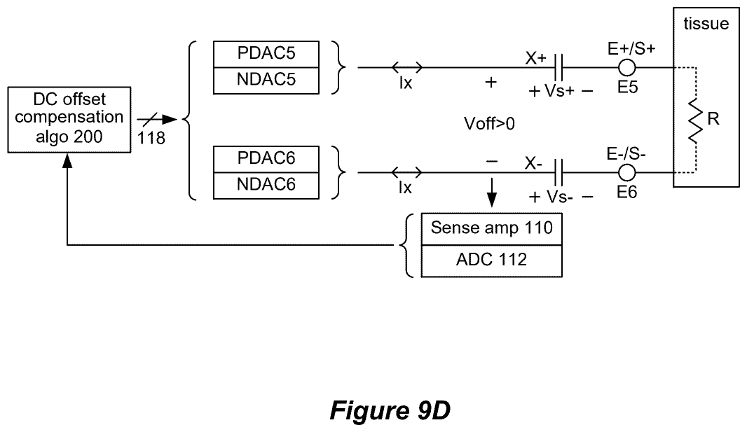

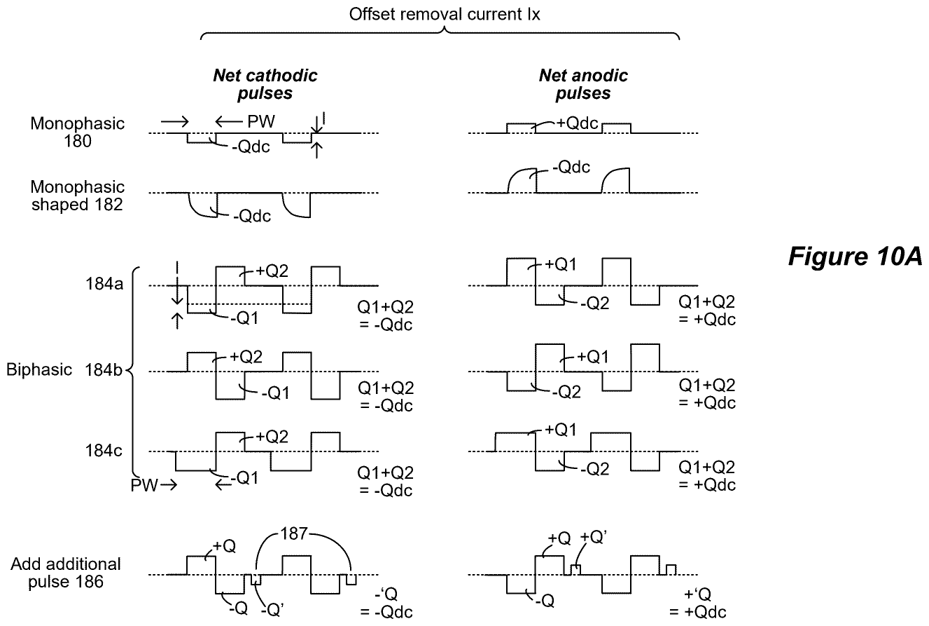

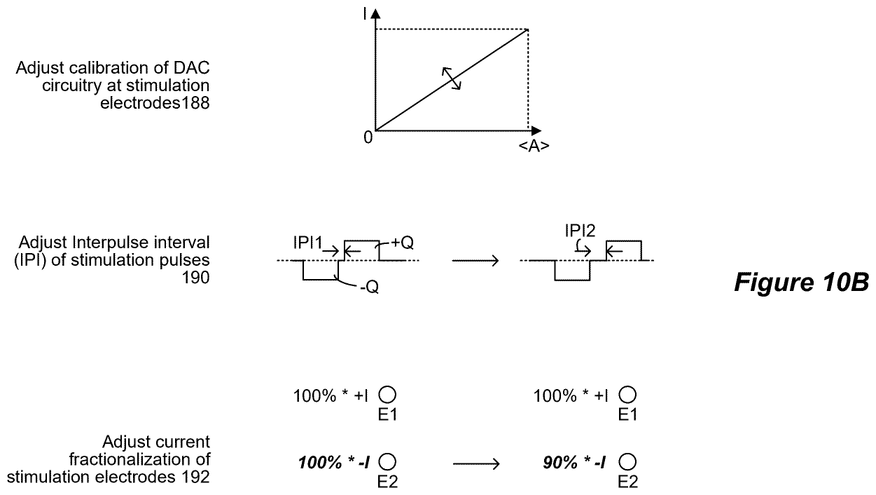

Prior to discussing examples of the DC offset algorithm, FIGS. 9A-10B show manners in which the algorithm can control the stimulation circuitry 28 to provide DC offset compensation at the inputs to the sense amp at the selected sensing electrode nodes. These examples assume as before (FIG. 7) that a stimulation current (Istim) is provided by the stimulation circuitry 28 at certain electrodes (E1, E2) in accordance with a stimulation program, and that a tissue signal such as a neural response will be sensed (differentially) at different electrodes (E5, E6). Istim may comprise a therapeutic stimulation current tailored to provide suitable stimulation therapy for the patient, which as noted earlier can comprise stimulation pulses with a prescribed amplitude, pulse width, frequency, etc. However, Istim can also comprise a non-therapeutic current (e.g., pulses) used specifically for the purpose of causing and measuring neural responses, which may be used in addition to therapeutic stimulation.

FIGS. 9A-9D show different examples of DC offset compensation, and in particular offset removal currents Ix that can be used to remove DC offsets. These currents Ix, as well as other compensation means described later (see FIGS. 10A, 10B) may be provided to the sensing electrodes S+/S−, any of the stimulating electrodes, or any other electrode.

FIG. 9A shows an example where offset removal current Ix is provided to one or more of the sensing electrodes S+/S−. In FIG. 9A it is assumed that DC offset Voff is greater than zero. To compensate, the DC offset compensation algorithm 200 can cause the stimulation circuitry 28 to issue an offset removal current Ix comprising one or more net cathodic pulses (−Q) at input X+ associated with sensing electrode S+ (E5), which can occur using NDAC5. This has the effect of decreasing Vs+ (e.g., eventually to the value of Vs−) and thus decreasing Voff closer to zero. Alternatively, or additionally, the DC offset compensation algorithm 200 can cause the stimulation circuitry 28 to issue as an offset removal current Ix one or more net anodic pulses (+Q) at input X− associated with sensing electrode S− (E6; using PDAC6). This has the effect of increasing Vs− (e.g., eventually to the value of Vs+), which also decreases Voff closer to zero.

The DC offset compensation algorithm 200 would operate similarly if Voff is less than zero, but would flip the polarities described above. In this circumstance, the DC offset compensation algorithm 200 can cause the stimulation circuitry 28 to issue an offset removal current Ix comprising one or more net anodic pulses at input X+ (using PDAC5), which would increase Vs+ and thus increase Voff closer to zero. And/or, the DC offset compensation algorithm 200 can cause the stimulation circuitry 28 to issue an opposite-polarity offset removal current Ix comprising one or more net cathodic pulses at input X− (using NDAC6), which would decrease Vs− and thus again increase Voff closer to zero.

Providing offset removal current Ix to the inputs X+/X− at the selected sensing electrodes S+/S− is beneficial because it directly corrects the DC offset Voff. However, DC offset compensation can also be provided by providing Ix to different electrodes. This method is less direct, but can affect and remove DC offset compensation by providing an electric field 202 whose presence is felt at the sensing electrode S+/S− through the tissue. For example, in FIG. 9B, the offset removal current Ix is provided at the one or more of the stimulation electrodes (E+/E−) that are also being used to provide the stimulation current, Istim. The additional electric field 202 created in the tissue (beyond any field stimulation Istim creates) by the offset removal current Ix, and which affects DC offset Voff, is shown generically. As explained further below, providing offset removal current Ix at the stimulation electrodes can occur either by causing the stimulation circuitry 28 at these electrodes to provide Ix in addition to Istim, or by modifying Istim to include Ix.

Offset removal current Ix can be provided to still other electrodes different from the stimulation electrodes (E+/E−) and the sensing electrodes (S+/S−). This is shown in FIG. 9C, where certain other electrodes (E3, E4) have been selected to provide the offset removal current, Ix (Y+/Y−). The electric field in the tissue that affects and reduces the DC offset Voff is again shown generically at 202.

FIG. 9D shows that the stimulation electrodes E+/E− can comprise the same electrodes as the sensing electrodes S+/S−, and with the offset removal current Ix (or other forms of compensation described shortly) applied to one or more of these common electrodes. Using the same electrodes for stimulation and sensing may be useful in some contexts, such as when detecting ERNA responses in a DBS context. Having said this, it is not essential that exactly the same stimulation and sensing electrodes be used, even in an ERNA context. Instead, there may be some overlap, in which some subset of electrodes is used both for stimulation and sensing. For example, although not illustrated, electrodes E4 and E5 could be used for stimulation, and electrodes E5 and E6 used for sensing, such that only electrode E5 is used for both stimulation and sensing. In this regard, it should be noted that the stimulation (E+/E−), sensing (S+/S−), and other electrodes (Y+/Y−) are not necessarily discrete from each other, and may partially or complete overlap in a particular application.

FIGS. 10A and 10B show different examples of offset removal currents Ix that can be used and formed by the stimulation circuitry 28. In one example, Ix can be formed of one or more pulses that are charge imbalanced, resulting in either a net anodic (+Qdc) or net cathodic (−Qdc) pulses at the electrodes. FIG. 10A provides various examples of charge imbalanced pulses that can be used to form Ix. Each of the pulses illustrated have a net charge of either −Qdc (net cathodic pulses) or +Qdc (net anodic pulses) depending on the polarity of currents used to produce them.

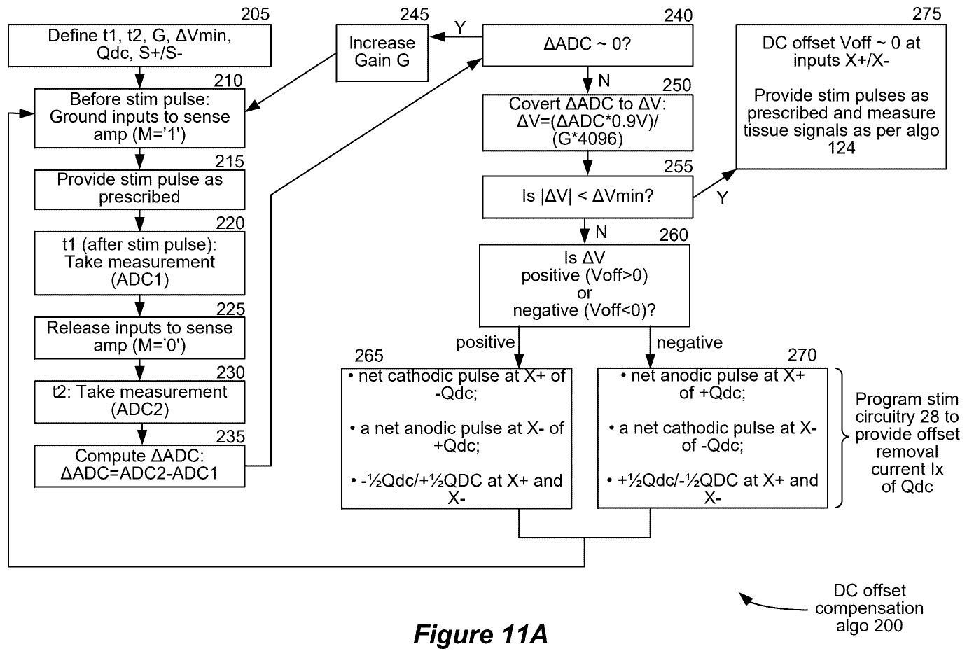

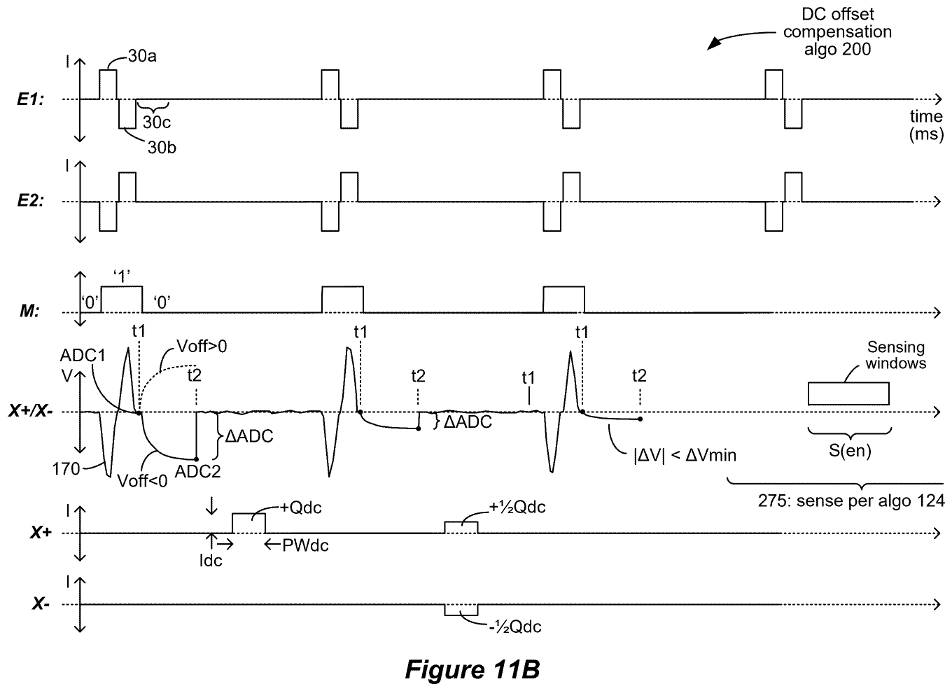

Pulses 180 are monophasic having only a single cathodic or anodic phase, in which Qdc is defined at the product of the pulse width (PW) and the current (I). FIG. 11B shows the use of such monophasic pulses 250 to provide the offset removal current Ix, as discussed later.

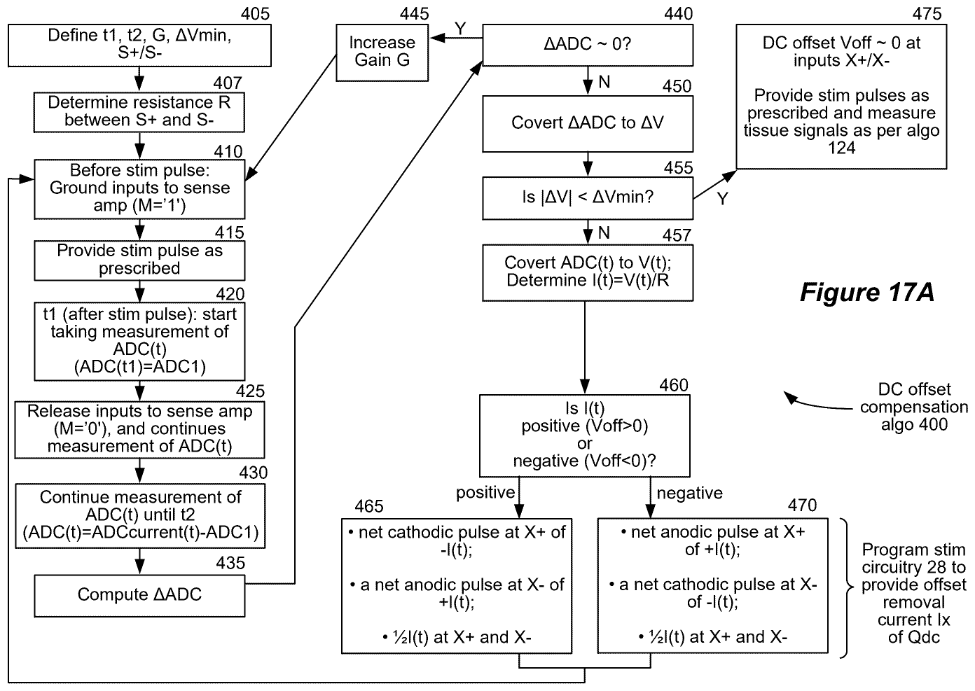

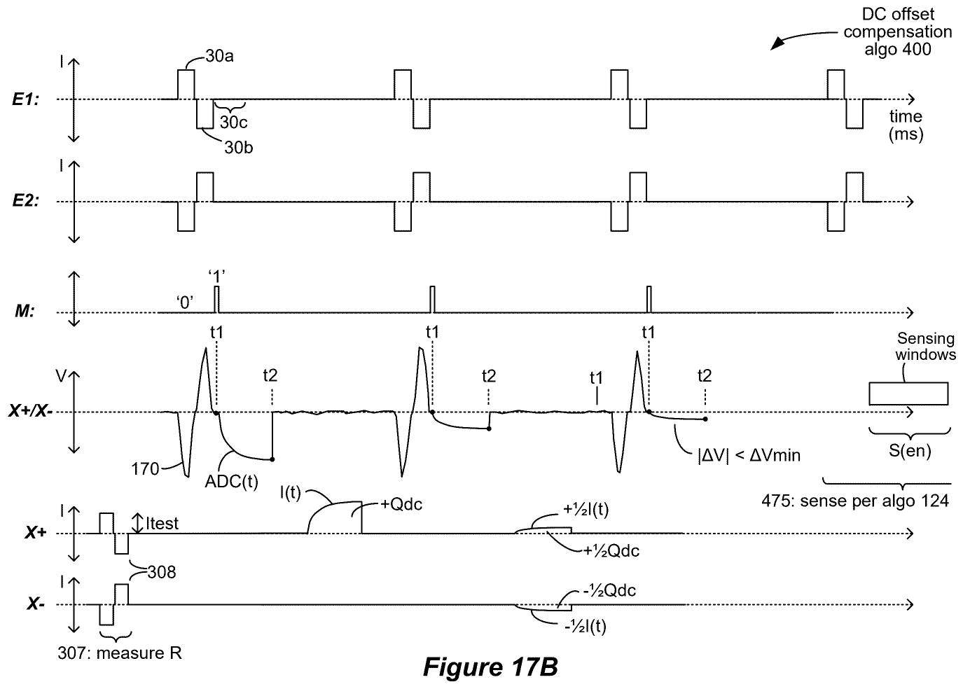

Pulses 182 are also monophasic, but are shaped, and in particular are shaped in a manner mimicking an RC (exponential) decay. As explained further with respect to FIGS. 17A and 17B, the use of such shaped pulses for the offset removal current Ix provides certain advantages.

The pulses can also comprise charge-imbalanced biphasic pulses. For example, biphasic pulses 184a and 184b have two phases with the same pulse width, PW, but with different amplitudes. For example, the net cathodic pulses have a cathodic phase (−Q1) with a larger amplitude than the anodic phase (+Q2, with the two phases differing by amplitude I), with the cathodic phases either occurring first (184a) or last (184b), resulting in a net cathodic charge of −Qdc. The net anodic pulses have an anodic phase (+Q1) with a larger amplitude than the cathodic phase (−Q2, again, differing by I), with the anodic phases either occurring first (184a) or last (184b), resulting in a net anodic charge of +Qdc. The biphasic pulses 184c have phases with the same current amplitude, but with different pulse widths, with the net cathodic pulses having a longer cathodic phase (−Q1), and with the net anodic pulses having a longer anodic phase (+Q1), which again results in net cathodic and anodic charges of −Qdc and +Qdc equal to the amplitude times the difference in the pulse widths of the phases.

Pulses 186 have an additional phase 187 (of charge +/−Q′) in addition to otherwise symmetric (charge balanced) biphasic pulses, leading to pulses that on the whole are charge imbalanced (+/−Qdc=Q′). This example may be particularly useful in circumstances where the stimulation electrodes (E+/E−) are used to provide Ix (FIG. 9B), because the symmetric biphasic pulses may provide Istim (and otherwise remain unaffected), with phase 187 added for charge imbalancing. These are just some examples of charge imbalanced pulses that the algorithm 200 can cause the stimulation circuitry 28 to produce to provide DC offset compensation. One skilled will recognize that other pulses of odd shapes and/or different numbers of phases are possible, and such approaches may comprise a combination, superposition, or summation of multiple monophasic, biphasic, or multiphasic pulses.

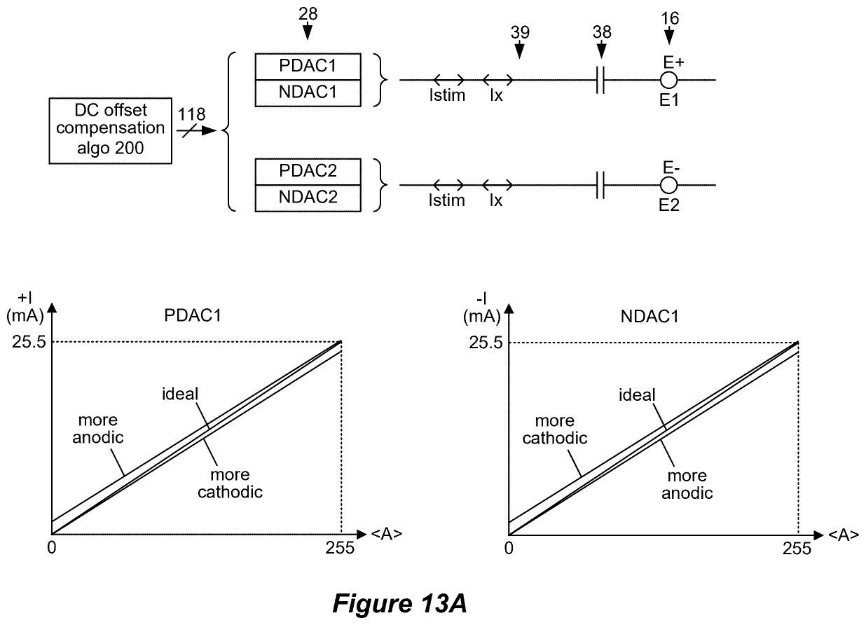

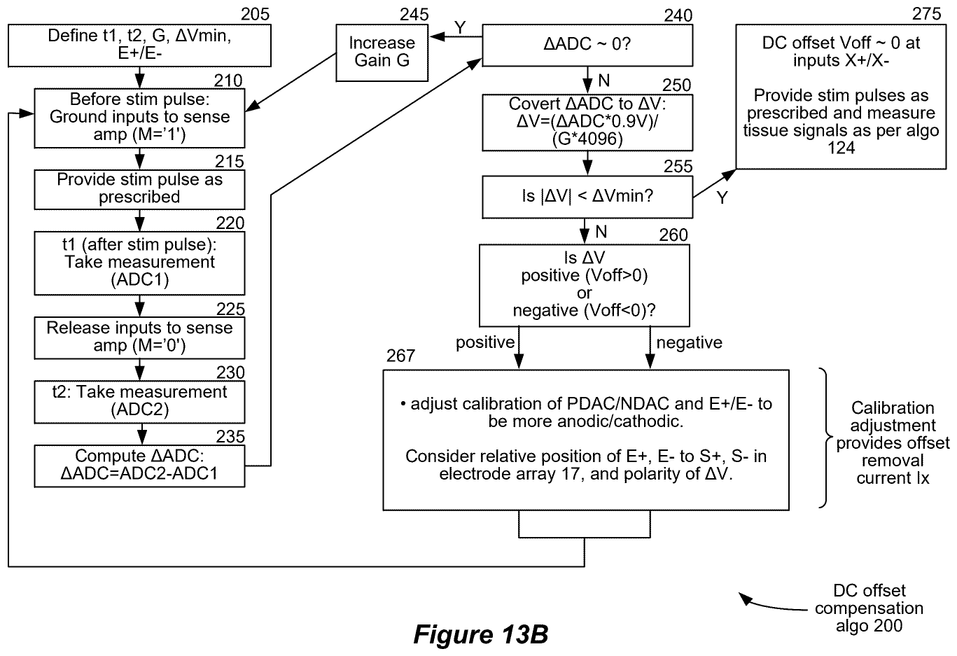

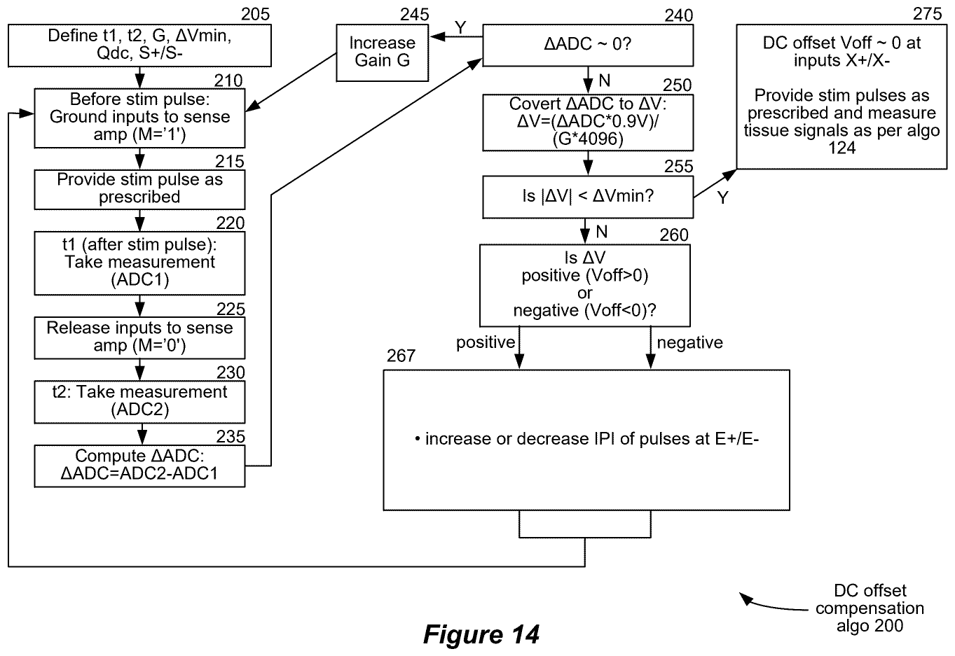

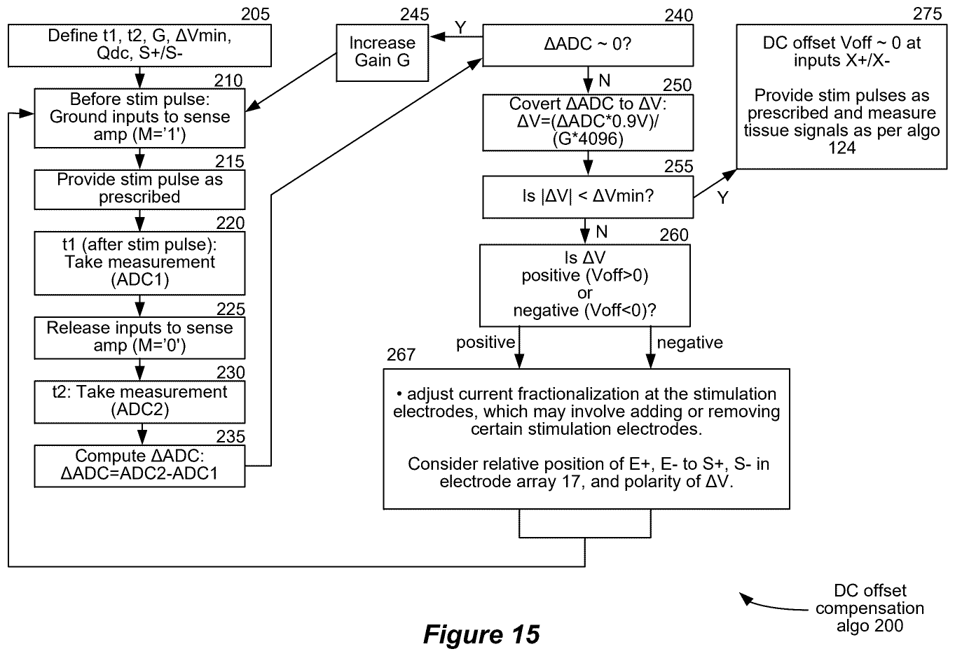

FIG. 10B shows other means by which the DC offset algorithm 200 can provide DC offset compensation. For example, and as discussed further below with respect to FIGS. 13A and 13B, the algorithm 200 can adjust the calibration of the DAC circuitry (the PDACs and NDACs) used to provide the stimulation current (188), thus making the currents output by the DACs more cathodic or more anodic. This calibration adjustment (changing the current) can comprise a representation of an offset removal current, Ix. In another example, and as discussed further below with respect to FIG. 14, the algorithm can adjust the interphase interval (IPI) between the phases in the biphasic pulse that normally forms the stimulation current, Istim (190). This interphase interval denotes a small time period during which no stimulation issues, allowing the stimulation circuitry 28 time to reconfigure itself after it has formed a pulse phase of one polarity, and before it forms the next pulse phase of the opposite polarity. FIG. 10B shows the simple example of an IPI adjustment from a first duration (IPI1) to a longer second duration (IPI2), although the IPI could also be made smaller. This example provides DC offset compensation without the use of an offset removal current Ix. In yet another example also not involving use of current Ix, and as discussed further below with respect to FIG. 15, the algorithm can adjust the manner in which current is fractionalized at the stimulation electrodes (192). Such fractionalization adjustment can involve steering some of the anodic (+I) or cathodic (−I) currents to different electrodes. Thus, in the simple example of FIG. 10B, some amount of the cathodic current (10%) has been steered from stimulation electrode E2 to a new stimulation electrode E3. All of these examples in FIG. 10B affect the field 202 produced in the tissue, which can be used to reduce the DC offset Voff, as explained further below.

FIG. 11A shows details of the DC offset compensation algorithm 200 in one example, with FIG. 11B comprising an accompanying timing diagram. In this example, the algorithm provides an offset removal current Ix directly to the inputs X+/X− of the sense amp coupled to the selected sensing electrodes.

As is true for all examples of the DC offset compensation algorithm 200 disclosed herein, one skilled in the art will understand that the order of steps could be varied, that additional steps could be added, or that certain steps can be removed. While the DC offset compensation algorithm 200 can operate on its own, it is preferably run automatically when the tissue signal sensing algorithm 124 (FIG. 5) is run. More particularly, when the sensing algorithm 124 is to be used to measure tissue signals at a given sense amp 110, algorithm 200 preferably runs first to reduce or remove any significant DC offset Voff that might be present at the input to that sense amp so that valid tissue signals can later be sensed by algorithm 124. Once any DC offset has been removed by algorithm 200, operation of the tissue signal sensing algorithm 124 can then run normally to sense the desired tissue signals during sensing windows (see, e.g., FIG. 11A, step 275). As such, operation of the DC offset compensation algorithm 200 may delay measurement and receipt of valid tissue signals as the DC offset is removed. Nevertheless, such delay would be relatively short (e.g., on the order of milliseconds), and thus would probably not be noticeable to or affect the therapy of the patient.

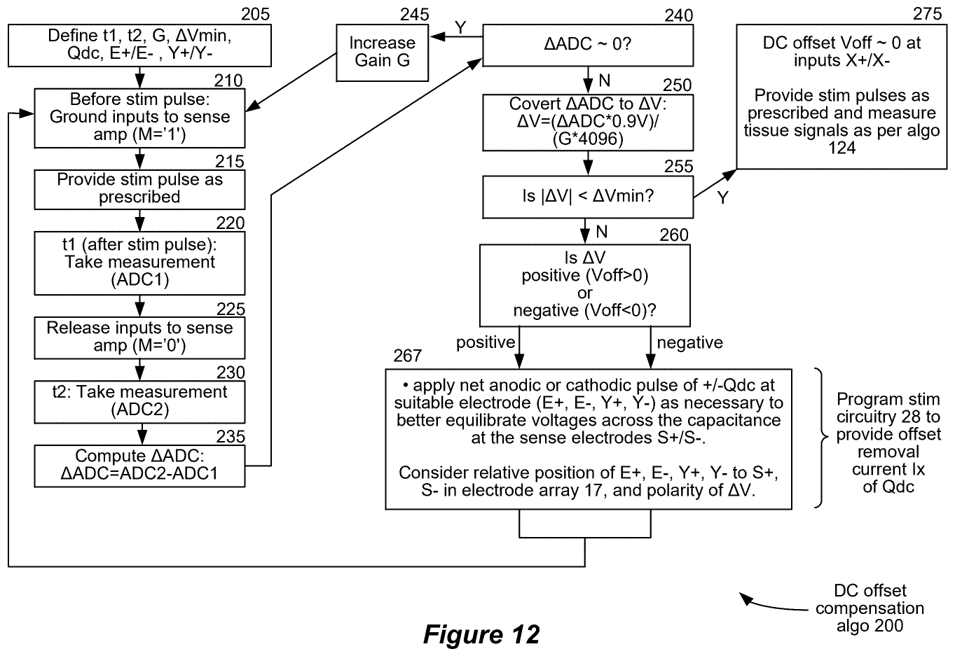

Step 205 defines certain parameters the algorithm 200 will use, which may be programmable or adjustable by the user. First, timing parameters t1 and t2 are specified which define the timing of measurements that are made by the algorithm 200, as explained further below. These timing parameters may for example vary in accordance with the frequency of the stimulation pulses, which can still be provided as the algorithm 200 operates. Parameter G comprises the gain of the sense amp 110 that is being used to take the measurements (and which will eventually be used for tissue signal sensing), which as noted earlier is programmable. As discussed further below, this gain G can be adjusted during the operation of the algorithm 200, and may initially be set to a low value (e.g., G=1). Parameter ΔVmin comprises a minimum acceptable value at the inputs X+/X− to the sense amp 110 below which (further) DC offset compensation is not needed. Also input at step 205 are the electrode(s) that will be used to provide DC offset compensation via offset removal current Ix. In this example, the sense electrodes (S+/S−) will receive Ix, but as noted earlier, compensation may be provided by the stimulation electrodes (E+/E−) or other electrodes (Y+/Y−), as shown in the later example of FIG. 12.

Qdc defines a charge value for DC offset compensation pulses (comprising Ix) that the algorithm 200 will cause the stimulation circuitry 28 to produce when reducing the DC offset Voff. Qdc may comprise a set value, which is designed to produce a particular reduction in Voff, and which may be applied iteratively by the algorithm 200 to gradually bring Voff toward zero. Alternatively, Qdc may vary as the algorithm 200 operates. For example, and although not shown, Qdc may be updated by the algorithm 200 to vary as a function of the measurement (e.g., ΔV, explained further below), thereby providing higher compensation (a higher Voff adjustment) initially, and smaller compensation as the algorithm iterates.

As an initial step 210, the inputs X+/X− to the sense amp 110 are shorted together, and more preferably shorted together at a known potential such as ground. This can occur by closing switches (e.g., transistors) 160 connected to the inputs of the sense amp as shown in FIG. 6 (e.g., M=‘1’). In another example not shown, a single switch 160 can be used to short both inputs together (but not to ground or any other known potential). Because the inputs X+ and X− are shorted, notice that Voff is temporarily set to zero.

A prescribed stimulation pulse for the patient is then provided at the selected stimulation electrodes (e.g., E1 and E2) (215), and such pulses may already be occurring to provide the patient therapy before algorithm 200 has started. FIG. 11B shows the effect of the stimulation pulses at the differential input X+/X− to the sense amp 110, where a stimulation artifact 170 is seen resulting from the electric field produced in the tissue by the stimulation. While this stimulation artifact 170 is shown for completeness, it is not necessary for algorithm 200 to detect this.

Referring again to FIG. 11A, while the switches 160 are shorted, and after the issuance of the stimulation pulse, the sense amp 110 preferably takes a baseline measurement at time t1 (220). Because Voff is temporarily set to 0V at this time, the resulting measurement (ADC1) should comprise approximately 0.45V, which when digitized by the ADC 112 would be in the middle (800H) of the operating range of the ADC 112 (ADC1). While it is advisable to take a measurement at time t1 at this step because non-idealities may cause the measurement to vary from Voff=0V, this is not strictly necessary, and the algorithm 200 could instead simply assume that the Voff is in fact zero at this point. If a measurement occurs at time t1, this preferably occurs at the end of the duration when the switches are closed (M=‘1’), as shown in FIG. 11B.

At step 225, the inputs to the sense amp 110 are released and no longer shorted to ground via the transistors 160 (M=‘0’). This allows Voff to gradually reestablish itself (relative to Vcm) based on the unbalanced charges (e.g., Vs+ and Vs−) present at the inputs X+/X−. Thus, if DC offset Voff is negative, as is assumed in FIG. 11B, the voltage at the input to the sense amps X+/X− will begin to fall. Conversely, if DC offset Voff is positive, the voltage at the input to the sense amp 110 will rise, as shown in dotted lines. Notice that this falling or rising is exponential, depending on the RC time constant of the circuitry on the front end of the sense amp 110, such as the capacitances 38 at inputs X+ and X−, and the resistance of the tissue R between them (FIG. 9A).

At step 230, the sense amp 110 takes another measurement at time t2. By this time, and as Voff is reestablished, the voltage at the inputs X+/X− has fallen to a negative value indicative of Voff, which when digitized would comprise a lower value in the ADC 112 (ADC2). Preferably time t2 is set (step 205) at a proper delay relative to time t1 (or relative to the transition to M=‘0’ at step 225) such that Voff is at least partially reestablished at the inputs before a next stimulation pulse is issued.

At step 235, the algorithm 200 computes a difference ΔADC=ADC2−ADC1 between the measurements taken at times t1 and t2. If Voff is negative as has been assumed to this point, note that ΔADC would be negative. If a measurement was not taken at time t1 (because ADC1 can be assumed as 800H), ΔADC would instead be established using only the measurement at time t2. Notice that ΔADC may be expressed as some number of steps in the ADC 112. (If the ADC is 12-bit, there would be 2\hat{ }12=4096 total steps).

At step 240, the algorithm 200 determines whether ΔADC equals zero (or alternatively is below some minimum number of ADC steps). This is preferable because the gain G of the sense amp 110 may need adjustment to properly resolve ΔADC: if ΔADC˜0, and the gain G of the sense amp is low, there could still be an appreciable DC offset Voff requiring compensation that is not yet resolvable. As such, if this condition is met, the gain G of the sense amp 110 is increased (245), and the algorithm 220 iterates to repeat the steps described earlier (210-235). As the gain G increases, eventually an appreciable ΔADC will be determined at step 240 that is suitable for further analysis.

At step 250, ΔADC is converted to a difference in voltage ΔV indicative of Voff at the inputs of the sense amp 110. This conversion depends on the particulars of the ADC 112 (e.g., its operating range, the total number of steps) and the current gain G of the sense amp. Assuming an ADC 112 with a 0.9V operating range and 4096 steps, the equation set forth in FIG. 11A will work the necessary conversion.

At step 255, the algorithm 200 determines whether the absolute value of ΔV is less than ΔVmin as set earlier (step 205). By way of review, parameter ΔVmin sets a minimum value below which (further) DC offset compensation is not needed. In other words, once |ΔV|<ΔVmin, the algorithm 200 will consider that the inputs to the sense amp 110 are sufficiently DC balanced (Voff˜0); that no further DC offset compensation is required; and therefore that sensing of tissue signals can begin reliably. In other words, tissue signal sensing algorithm 124 can begin in earnest at this point to validly sense tissue signals, as shown at step 275.

If |ΔV| is not less than ΔVmin, the algorithm 200 concludes that (further) DC offset compensation is needed, and first determines whether ΔV (and hence Voff) is positive or negative (260). If negative, as is assumed in FIG. 11B, the algorithm 200 proceeds to step 270. At this step, the algorithm 200 causes the stimulation circuitry 28 to provide an offset removal current Ix in the form of a pulse with a charge equal to Qdc (as defined earlier in step 205). This can occur in different manners. As shown in FIG. 11B, a net anodic pulse of +Qdc can be provided to input X+, and assuming electrode E5 operates as S+, this pulse would be provided by controlling the PDAC at this electrode (PDAC5). Providing this net anodic pulse would increase Vs+ across the capacitor at X+, which would increase Voff closer to zero. In FIG. 11B, the net anodic pulse is shown as a monophasic pulse, as was shown at 180 in FIG. 10A, but shaped (182), biphasic (184) net anodic pulses could be used, as well as pulses having additional phases (186). Alternatively, step 270 could provide a net cathodic pulse of −Qdc at the other input X− (using NDAC6 associated with electrode E6). This would decrease Vs− at the capacitor of X−, which again would increase Voff closer to zero. Still further, the offset removal current Ix can be provided at both of these inputs X+ and X−, with Qdc being split (e.g., +½ Qdc and −½Qdc) and provided to each. (FIG. 11B shows this alternative after the second pulse). Providing a current to both inputs is especially useful if a current return (e.g., Vcm) has not already been established in the tissue.

The algorithm 200 has some discretion in controlling the stimulation circuitry 28 to issue the offset removal current Ix in accordance with Qdc. Assuming that the stimulation circuitry 28 is controllable to issue a constant current, the algorithm 200 can choose a pulse width (PWdc) and amplitude (Idc) for the compensation pulse such that PWdc*Idc=Qdc, meaning that PWdc and Idc, while related, can vary. Further, and as noted earlier, Qdc can be made to vary as the algorithm 200 iterates, and can be a function of ΔV for example, such that a smaller Qdc is applied as compensation as ΔV gets closer to ΔVmin.

If ΔV is instead positive at step 260, the algorithm 200 proceeds to step 265, which provides a charge pulse Qdc with the opposite polarity when compared to step 270. Thus a net cathodic pulse of −Qdc can be provided at X+ (e.g., using NDAC5); a net anodic pulse of +Qdc can be provided at X− (using PDAC6); or −½ Qdc and +½Qdc can be provided to both, all of which cause Voff to decrease.

At this point, the algorithm 200 can repeat, by repeating the measurement and assessment steps described earlier (210-260). Because Voff has been partially compensated for at this point (265, 270), notice in FIG. 11B that the next resulting measurement ΔADC is smaller in magnitude. Nevertheless, the algorithm 200 can continue to iterate until such time as |ΔV| is smaller than ΔVmin (255). As described earlier, at this point, Voff is sufficiently close to zero that further DC offset compensation (265, 270) is not required, and valid tissue signal sensing can begin ay a sensing window (275).

FIG. 12 shows another example of DC offset compensation algorithm 200, which differs in that the offset removal current Ix is not applied directly to the inputs X+/X− at the sensing electrodes S+/S−. Instead, in this example, current Ix is provided either by the stimulation electrodes E+/E− (FIG. 9B), or some other electrodes Y+/Y− (FIG. 9C), which as discussed earlier, makes an electric field 202 in the tissue to provide compensation at inputs X+/X− to the sense amp. The particular electrodes which will receive offset removal current Ix can be programmed at step 205, or the algorithm 200 can automatically determine the electrodes to be used, which may depend on which stimulation and sensing electrodes have been chosen, and the relative position of the electrodes in the electrode array 17. For example, the algorithm 200 may select to use electrodes Y+/Y− for DC offset compensation that are close to the sensing electrodes S+/S−.

The algorithm 200 of FIG. 12 includes the same initial steps discussed earlier (210-260), but differs at step 267 which applies the offset removal current, Ix. The algorithm 200 can control the stimulation circuitry 28 at step 267 to provide a current Ix which produces a field 202 (FIGS. 9B, 9C) which will tend to remove the imbalance, whether it is positive or negative. The algorithm 200 preferably considers the positions of the electrodes in the electrode array 17 when making adjustment at step 267, as well as the polarity of Voff. For example, assuming that other electrodes E3/E4 (Y+/Y−) are used to apply Ix (FIG. 9C), and if Voff is positive, the algorithm 200 can cause a net anodic pulse to issue at Y− (E4). This pulse would tend to create voltage at E4 which is higher than Vcm, and thus an electric field 202 in which the voltage will decrease (down to Vcm) as the distance from E4 increases. Thus, electric field 202 will create a generally higher voltage (relative to Vcm) at E5 (S+) which is closer to E4, and smaller voltage at E6 (S−) which is farther away from E4. Presenting these voltages via the field 202 to the sensing electrodes in this manner would tend to equate the voltages across the capacitors at the sensing electrodes, thus reducing DC offset Voff. Providing a net anodic pulse at Y− could be accompanied by a net cathodic pulse at Y+ (e.g., E3).

Conversely, if Voff is negative, applying a net cathodic pulse at Y− would tend to create voltage at E4 which is lower than Vcm, and thus an electric field 202 in which the voltage will increase (up to Vcm) as the distance from E4 increases. Thus, electric field 202 will create a generally lower voltage at closer electrode E5 (S+), and higher voltage at farther electrode E6 (S−). This would also tend to equilibrate the voltages across the capacitors at the sensing electrodes.

The electrodes used to provide the stimulation (E+/E−) can also be used to provide Ix. As noted earlier, Ix can be formed at the stimulation electrodes to form net anodic or cathodic pulses either by modifying the phases of the stimulation pulses providing Istim or by adding an addition phase (e.g., 187, FIG. 10A) to Istim. Again, the polarity of Voff and relative position of the stimulation electrodes to the sensing electrodes in the electrode array 17 may need to be considered so that Ix forms a field 202 which tends to remove Voff, as described above. As before, the algorithm 200 of FIG. 12 can continue to iterate until further DC offset compensation is no longer required (at step 255).

The algorithm 200 of FIGS. 13A and 13B differs in that DC offset compensation occurs through adjustment of calibration of the stimulation circuitry 28, and in particular the calibration of the stimulation circuitry active at the stimulation electrodes E+/E− to provide the stimulation current, as was described earlier with respect to FIG. 10B (188). In this example, the offset removal current Ix results from adjusting the PDACs or NDACs in the stimulation circuitry 28 that produce the stimulation current Istim to make that stimulation current more or less cathodic or anodic. As in the example just discussed, such calibration can be employed to affect the electric field 202 produced in the tissue in a manner that reduces or removes the DC offset Voff.

As shown in FIG. 13A, the algorithm 200 can control the stimulation circuitry 28 via bus 118, which includes signals necessary for calibration of the PDACs and NDACs at the selected stimulation electrodes. Calibration of the PDAC and NDAC circuitry can occur as explained in U.S. Pat. No. 11,967,969, which is incorporated herein by reference in its entirety, and which is briefly explained with reference to FIG. 13A. As explained in the '969 patent, control signals (118) can adjust each PDAC and NDAC individually, and in so doing can change the value of the anodic and cathodic currents each respectively produces. This is shown graphically, where <A> represents the digital amplitude bus (also part of bus 118) that is used to set the amplitude each DAC produces. Current I represents the actual current the DAC produces in response to the value of <A>. In an ideal case, each DAC would produce 0 mA when <A>=0, and a maximum current (e.g., +/−25.5 mA) when <A> is maximized. Assuming <A> represents an 8-bit amplitude bus, this bus would be maximized when <A>=11111111, or 255 in decimal, such that incrementing the value on the amplitude bus <A> would increase output current I in 0.1 mA increments.

Calibration as controlled by DC offset algorithm 200 via bus 118 however can change the current produced by the DACs away from this ideal. For example, the PDAC at electrode E1 (PDAC1) can be calibrated to produce anodic currents (+I) that are higher than ideal (more anodic), or lower than ideal (more cathodic) as shown. Likewise, the NDAC at electrode E1 (NDAC1) can be calibrated to produce cathodic currents (−I) that are higher than ideal (more cathodic), or lower than ideal (more anodic).

As such, the DC offset algorithm 200 as shown in FIG. 13B can control the DACs that produce the stimulation current to modify them in a way (Ix) which makes them more or less cathodic, which can reduce the DC offset Voff at the sensing electrodes. As before, the algorithm 200 preferably considers the polarity of Voff and the relative positions between the stimulation and sensing electrodes in the electrode array 17 when making calibration adjustment at step 267. For example, if Voff is positive, the algorithm 200 can adjust the calibration at PDAC2 and/or NDAC2 at electrode E2 (E−) to create currents that are more anodic. Stimulation pulses at E2 would thus tend to create voltages at E2 which are higher, and in turn electric field 202 will create a generally higher voltage (relative to Vcm) at E5 (S+) which is closer to E2, and a smaller voltage at E6 (S−) which is farther away from E2. Presenting these voltages via the field 202 to the sensing electrodes would tend to equate the voltages across the capacitors at the sensing electrodes, thus reducing DC offset Voff as explained earlier. Conversely, if Voff is negative, the algorithm 200 can adjust the calibration at PDAC2 and/or NDAC2 at electrode E2 (E−) to create currents that are more cathodic. Note that the calibration adjustment at step 267 does not seek to necessarily apply a particular amount of net charge, such as Qdc.

Notice that DC offset algorithm 200 of FIGS. 13A and 13B adjusts the stimulation current (Istim) from what was otherwise specified to treat the patient. As such, the calibration adjustment this algorithm 200 employs has the potential to affect patient therapy. Having said this, calibration does not typically greatly change the currents at the stimulation electrodes, and as such it would not be expected that use of this example of the algorithm 200 would produce a noticeable effect on patient therapy.

The algorithm 200 of FIG. 14 does not affect DC offset compensation by providing an offset removal current, Ix. Instead, this example of the algorithm adjusts the interpulse interval (IPI) of the stimulation pulses issued at the stimulation electrodes, as was described earlier with respect to FIG. 10B (190). Adjustment of the IPI can affect the field 202 produced in the tissue, which in turn can reduce the DC offset Voff as described earlier. The IPI is generally short in comparison to the actively-driven pulse phases in a biphasic pulse. As such, the electric fields produced in the tissue by the first phase may not have completely dissipated by the time the second phase arrives (particularly if the IPI is small), leading to uncontrolled charge flow during the IPI when charge is not actively driven. As such, changing the IPI (267) will change this uncontrolled charge flow, and thus the net field 202 produced in the tissue by the biphasic pulse. How the algorithm 200 can adjust the duration of the IPI at step 267 to reduce the DC offset Voff may require experimentation. In one example, the algorithm 200 can iteratively increase (or decrease) IPI to see its effect on the DC offset. This may eliminate the DC offset Voff, or make it worse. If it is worse, the algorithm 200 can instead decrease (or increase) the IPI as it iterates.

The algorithm 200 of FIG. 15 at step 267 adjusts the current fractionalization of the stimulation pulses, as was described earlier with respect to FIG. 10B (192). Fractionalization cause some amount of anodic (+I) or cathodic (−I) current to be steered between the stimulation electrodes. Such steering can involve a redistribution of the anodic or cathodic currents between already existing stimulation electrodes. For example, if stimulation electrodes E1, E2 and E3 are initially receiving 50%*+I, 50%*+I, and 100%*−I respectively, such steering can involve changing the currents at these electrodes to 40%*+I, 60%*+I, and 100%*−I. Steering can also involve providing current to new stimulation electrodes (such as E3 in FIG. 10B), or the removal of a stimulation electrode. Steering of the stimulation current in this fashion will change the field 202 produced in the tissue, which in turn will affect the DC offset Voff. In this regard, and as in other examples, how the current can be steered by the algorithm 200 can depend on the polarity of Voff as reflected by ΔV and relative position of the stimulation electrodes to the sensing electrodes in the electrode array 17. If Voff is positive for example, the algorithm 200 may steer some portion of the anodic current to an electrode (e.g., E3) that is closer to E5 (S+). This adjusts the field 202 to produce higher voltages at E5 (S+) and a lesser voltage at E6 (S−), which as noted earlier would tend to reduce this offset. If Voff is negative, the algorithm 200 may instead steer some portion of the cathodic current to that electrode (e.g., E3).

The various techniques by which the DC offset algorithm 200 can make adjustments to reduce or eliminate DC offset Voff as shown in FIG. 11A-15 are not mutually exclusive, and other examples of the algorithm 200 can involve the use of more than one of these techniques. For example, certain of the disclosed techniques can be used for a coarse adjustment to Voff (when Voff is large), while others may be used for fine adjustments (when Voff is smaller).

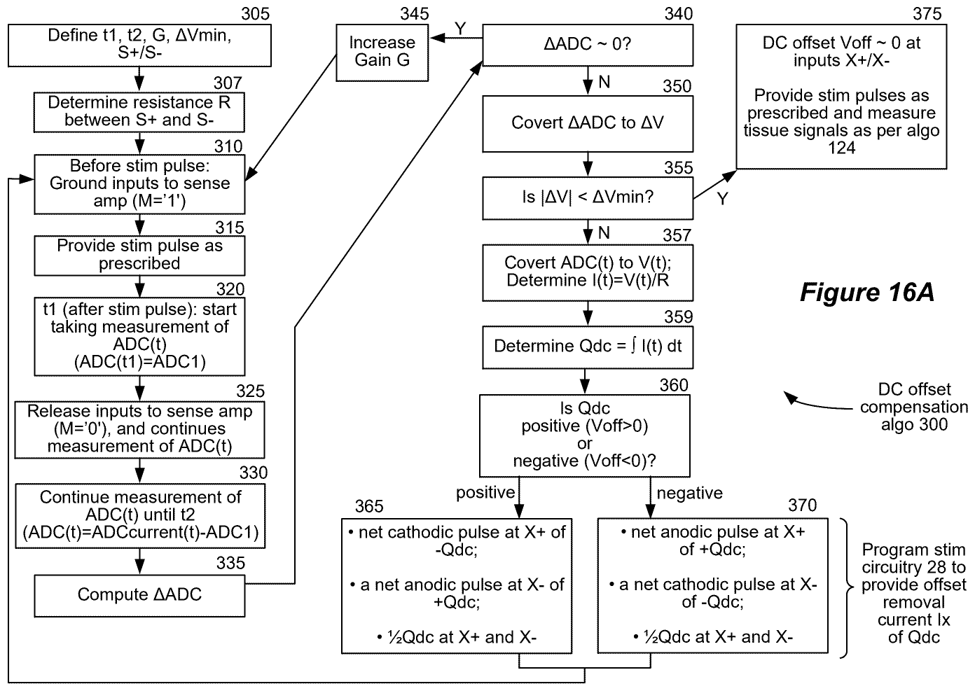

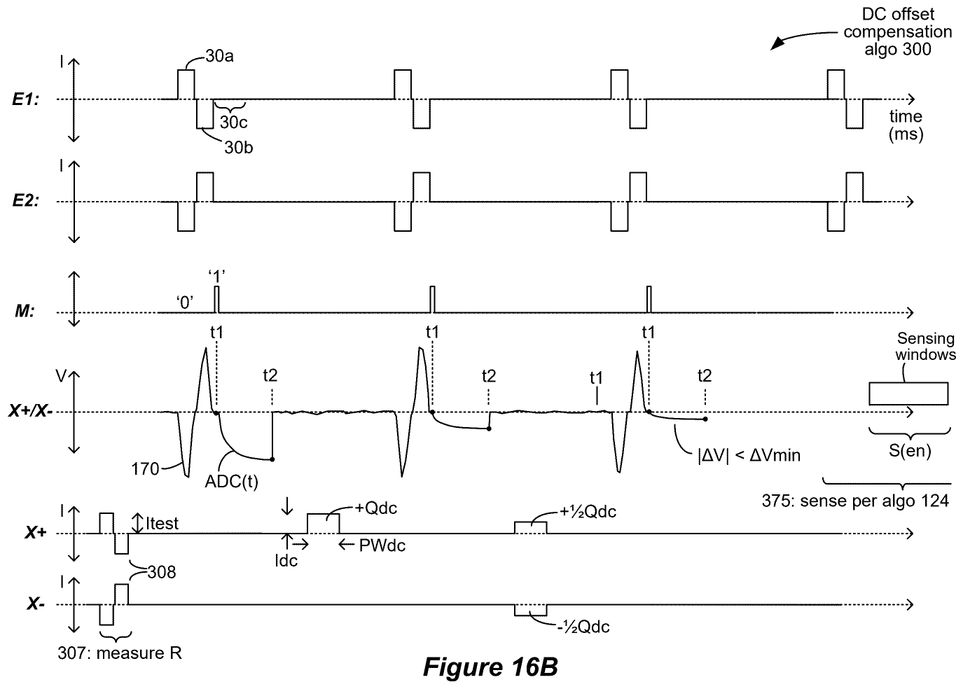

FIGS. 16A and 16B show another example of DC offset compensation algorithm 300. This example is different in that Qdc is determined as the algorithm 300 operates. This allows algorithm 300 to calculate an amount of compensation that is needed and applied via the offset removal current Ix. Because Qdc is calculated, DC offset compensation is potentially achieved after the application of a single compensation pulse, or is achieved much more quickly. Additionally, the measurement made by algorithm 300 is not based on measurements indicative of Voff at discrete points in time (e.g., at just t1 and t2). Instead, algorithm 300 measures the resulting waveform indicative of Voff as a function of time, using this waveform to determine Qdc. Because Qdc is determined, Qdc is not predefined at step 305, although other parameters discussed earlier may be. In this example, the offset removal current Ix is preferably applied directly to the sense amp inputs X+/X− at the sensing electrodes S+/S−. However, this is not strictly required, and Ix could be applied at the stimulation electrodes (E+/E−) or other electrodes (Y+/Y−) as described in other examples.

In step 307, the resistance between the selected sensing electrodes S+ and S− is measured, which will be dominated by the resistance of the tissue between these electrodes (R, FIG. 9A). As one skilled in the art will recognize, this resistance R can be determined in different manners. In one example, the algorithm 300 causes the stimulation circuitry 28 to issue a test pulse 308 of a known current amplitude Itest at the inputs X+ and X− corresponding to S+ and S−, as shown in FIG. 16B. Tissue resistance circuitry 165 (FIG. 5) receives the voltages at the electrodes that result from the test pulse, and determines R accordingly. In the example shown, the test pulse 308 is biphasic, which advantageously allows the effect of the capacitors 38 to be canceled out of the measurement of R, as explained for example in U.S. Pat. No. 9,061,140. This patent shows sample and hold circuitry and related measurement details that can be used for tissue resistance circuitry 165, and is incorporated herein by reference in its entirety, but again the tissue resistance R can be determined in other manners and using different circuitry. The test pulse 308 (the resistance measurement more generally) can occur during periods when the stimulation circuitry 28 is not otherwise providing the stimulation pulses (at electrodes E1, E2), as shown in FIG. 16B. Alternatively, the test pulse 308 for the resistance measurement can be made by controlling the stimulation circuitry 28 in a different timing channel from that used to issue the stimulation pulses.

Steps 310-355 largely mimic steps 210-255 described earlier, but are modified so that data indicative of the DC offset Voff is measured as a function of time. As before, the inputs to the sense amp are grounded (M=‘1’; 310), and a stimulation pulse is again provided (315). At time t1 (when M=‘1’; 320), the sense amp 110 and ADC 112 start measuring to capture an output as a function of time (ADC(t)), which preferably includes an initial value (ADC(t1)=ADC1) operating as a baseline. (As explained earlier, it would be expected that Voff˜0, and that that ADC1˜0.45V or 800H at the ADC 112).

Thereafter (325), the inputs X+/X− to the sense amp 110 are released (M=‘0’), which as explained earlier causes Voff to at least partially reestablish at the inputs. This causes (as assumed in FIG. 16B) Voff to fall, and this fall continues to be monitored at the ADC 112 (330) until time t2 to determine the output of the ADC as a function of time (ADC(t)). Note that ADC(t) as determined at this step may comprise the current value of the ADC (ADCcurrent(t)) minus the background value measured by the ADC at t1 (ADC1). As such, ADC(t) would in this example comprise negative values (steps).

Steps 335-355 are largely similar to steps 235-255 described previously, and are used to determine whether the gain G of the sense amp 110 should be adjusted, and whether DC offset compensation is complete. Thus, ΔADC is calculated (e.g., ADC(t2)−ADC1(t1); 335) and assessed (340) to determine whether sense amp gain G adjustment (345) is required. If not, ΔADC can be converted to ΔV (350), with |ΔV| then being compared to ΔVmin to determine whether further DC offset compensation is needed (355), as described earlier. If |ΔV|<ΔVmin and therefore no (further) DC offset compensation is required, sensing of tissue signals may begin in earnest (375).

If further DC offset compensation is required, ADC(t) is converted to a voltage (V(t)) (357), which can comprise use of the equation explained earlier (see FIG. 11A, 250). Further at this step, this voltage V(t) is converted to a current (I(t)) using the tissue resistance R determined earlier (307). Qdc is then determined as the integral of I(t) (359), and in effect comprises the area under curve I(t). Note in this example (Voff<0) that V(t), I(t), and Qdc would be negative. Although not shown, realize that the computation of Qdc can also be used to determine whether DC offset compensation is complete in lieu of step 355 which considers ΔV. Thus, a minimum Qdc (Qdc(min)) can be predefined at step 305, and compared to |Qdc| as calculated at step 359 to determine whether DC offset compensation is complete (i.e., whether the algorithm 300 can proceed to step 375).

Once Qdc's polarity is determined (360), the algorithm 300 proceeds to steps 360 (positive) or 370 (negative) to apply a compensating pulse (Ix) with charge Qdc. As before, this pulse can be provided either of the inputs X+/X−, or both, depending on the polarity. The algorithm 200 again has some discretion in controlling the stimulation circuitry 28 to issue the compensating pulse in accordance with Qdc, and for example can choose a pulse width (PWdc) and amplitude (Idc) accordingly as described earlier.

The algorithm 200 can then repeat (315-360), and providing additional compensation (360, 370) as necessary. Note that further compensation may not be required. This is especially true if Voff was provided enough time to fully reestablish (at step 325) because time t2 is suitably far out in time. Nevertheless, algorithm 300 seeks to determine and apply the amount of DC offset compensation that is required, causing compensation to occur more quickly.

FIGS. 17A and 17B show another example of DC offset compensation algorithm 400. This example is different in that the charge of compensation pulses, Qdc, is not predefined or determined. Instead, compensation is provided by controlling the stimulation circuitry 28 to issue a charge-imbalanced monophasic shaped pulse as the offset removal current Ix, similar to that shown earlier (FIG. 9B, 182). This shaped pulse varies as a function of time similarly to the manner in which ADC(t) is shaped. In effect, the shaped pulse applies Qdc as occurred in algorithm 300 without the need to specifically calculate it. In this example, the offset removal current Ix comprising the shaped pulse is preferably applied directly to the sense amp inputs X+/X− of the sensing electrodes S+/S−. However, this is again not strictly required, and Ix could be applied at the stimulation electrodes (E+/E−) or other electrodes as described in other examples.

Steps 405-457 are the same as steps 305-357 described earlier, culminating in the determination of time-varying current I(t). Next step 460 determines whether I(t) is positive (Voff >0) or negative (Voff<0). At next steps 465 and 470, current I(t) once determined is simply applied as a shaped pulse at one or both of inputs X+ and X− to provide DC offset compensation. Notice as shown in FIG. 17B, and by virtue of the manner in which it is determined, that I(t) matches the shape of ADC(t) (and hence V(t)), and thus matches the shape of Voff as it is reestablished after time t1. Applying I(t) as a compensation pulse in this manner applies a compensating charge of Qdc just as occurred in algorithm 300, but without the need to integrate to calculate Qdc, and without the need to specifically determine a new pulse with charge Qdc.

Because the compensation pulses in this example are time-varying and not of constant current as in other examples, it may be more difficult for the stimulation circuitry 28 to provide these pulses under control by the algorithm 400. Nevertheless, the art describes architectures and simulation circuitry that can be used to easily provide such time-varying pulses. See, e.g., U.S. Pat. No. 10,576,265, which is incorporated herein by reference in its entirety.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for operating a stimulator device comprising a plurality of electrode nodes, wherein each of the electrode nodes is associated with a different electrode configured to contact a patient's tissue, the method comprising:
providing from stimulation circuitry stimulation to one or more first of the plurality of electrode nodes to provide stimulation to the patient's tissue;
using sense amplifier circuitry to sense a tissue signal, the sense amplifier circuitry comprising a first input and a second input, wherein the sense amplifier circuitry receives second of the plurality of electrode nodes at the first and second inputs;
shorting and then releasing the first and second inputs, and receiving at least one measurement from the sense amp circuitry taken after the release to produce data indicative of a DC offset voltage between the first input and the second input; and
using the data to control the stimulation circuitry to reduce or eliminate the DC offset voltage.

2. The method of claim 1, wherein a DC-blocking capacitor is between each of the electrode nodes and its associated electrode.

3. The method of claim 1, wherein the data is used to control the stimulation circuitry to reduce or eliminate the DC offset voltage by issuing an offset removal current at one or more of the electrode nodes.

4. The method of claim 3, wherein the offset removal current comprises one or more charge imbalanced pulses.

5. The method of claim 3, wherein the offset removal current is issued at one or more of the one or more first electrode nodes.

6. The method of claim 5, wherein the data is used to control the stimulation circuitry to reduce or eliminate the DC offset voltage by adjusting a calibration of the stimulation circuitry.

7. The method of claim 3, wherein the offset removal current is issued at one or more of the one or more second electrode nodes.

8. The method of claim 3, wherein the offset removal current is issued at one or more of the plurality of electrode nodes other than the first or second electrode nodes.

9. The method of claim 1, wherein at least one of the one or more first electrode nodes is the same as at least one of the one or more second electrode nodes.

10. The method of claim 1, wherein at least a portion of the method is controlled by an algorithm programmed into control circuitry of the stimulator device.

11. The method of claim 10, wherein the algorithm iterates by periodically producing the data indicative of the DC offset voltage, and periodically uses the data to control the stimulation circuitry to reduce or eliminate the DC offset voltage.

12. The method of claim 1, wherein the stimulation is provided as biphasic pulses, and wherein the data is used to control the stimulation circuitry to reduce or eliminate the DC offset voltage by adjusting an interphase interval between the phases of the biphasic pulses.

13. The method of claim 1, wherein the data is used to control the stimulation circuitry to reduce or eliminate the DC offset voltage by adjusting an anodic or cathodic current of the stimulation at the one or more first electrode nodes.

14. The method of claim 13, wherein the adjustment of the anodic or cathodic current comprises steering at least some of anodic or cathodic current between the one or more first electrode nodes.

15. The method of claim 13, wherein the adjustment of the current comprises steering at least some of anodic or cathodic current to a new one or more first electrode nodes.

16. The method of claim 1, wherein the first and second inputs are shorted to a reference voltage.

17. The method of claim 1, further comprising receiving a first measurement from the sense amp circuitry taken while the first and second inputs are shorted, and a second measurement taken after the release, wherein the data indicative of the DC offset voltage comprises a difference between the first and second measurements.

18. The method of claim 1, wherein the method receives a plurality of measurements over time from the sense amp circuitry taken after the release, wherein the data indicative of the DC offset voltage varies over time.

19. A stimulator device, comprising:
a plurality of electrode nodes, wherein each of the electrode nodes is associated with a different electrode configured to contact a patient's tissue;
stimulation circuitry configurable to provide stimulation to one or more first of the plurality of electrode nodes to provide stimulation to the patient's tissue;
sense amplifier circuitry comprising a first input and a second input, wherein the sense amplifier circuitry is configurable to receive second of the plurality of electrode nodes at the first and second inputs, wherein the sense amplifier circuitry is configured to sense a tissue signal; and
control circuitry configured to short and then release the first and second inputs, and to receive at least one measurement from the sense amp circuitry taken after the release to produce data indicative of a DC offset voltage between the first input and the second input;
wherein the control circuitry is further configured to use the data to control the stimulation circuitry to reduce or eliminate the DC offset voltage.

20. A non-transitory computer readable medium comprising instructions executable in a stimulator device comprising a plurality of electrode nodes, wherein each of the electrode nodes is associated with a different electrode configured to contact a patient's tissue, wherein the instructions when executed are configured to:
provide from stimulation circuitry stimulation to one or more first of the plurality of electrode nodes to provide stimulation to the patient's tissue;
use sense amplifier circuitry to sense a tissue signal, the sense amplifier circuitry comprising a first input and a second input, wherein the sense amplifier circuitry is configurable to receive second of the plurality of electrode nodes at the first and second inputs;
short and then release the first and second inputs, and receive at least one measurement from the sense amp circuitry taken after the release to produce data indicative of a DC offset voltage between the first input and the second input; and
use the data to control the stimulation circuitry to reduce or eliminate the DC offset voltage.
